(19) United States
(12) Patent Application Publication
(10) Pub. No.: US 2008/0227823 A1
(43) Pub. Date: Sep. 18, 2008

(54) AMIDE DERIVATIVES AS CALCIUM CHANNEL BLOCKERS

(76) Inventors: Hassan Pajouhesh, West Vancouver (CA); Nagasree Chakka, Burnaby (CA); Yuanxi Zhou, Richmond (CA); Francesco Belardetti, Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 12/042,277

(22) Filed: Mar. 4, 2008

Related U.S. Application Data
(60) Provisional application No. 60/894,392, filed on Mar. 12, 2007.

(51) Int. Cl. A61K 31/4439; C07D 401/12; C07D 209/04; A61K 31/404; C07D 211/06; A61K 31/4468

(52) U.S. Cl. 514/339; 546/277.4; 548/491; 514/415; 546/309; 514/352

(57) ABSTRACT

Methods and compounds effective in ameliorating conditions characterized by unwanted calcium channel activity, particularly unwanted T-type calcium channel activity are disclosed. Specifically, a series of compounds containing both a diamide and aromatic functionality are disclosed of the general formula (1) where X¹ and X² are linkers.

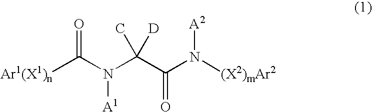

### CROSS-REFERENCE TO RELATED APPLICATIONS

This application claims benefit of Provisional Application Ser. No. 60/894,392, filed 12 Mar. 2007. The contents of these documents are incorporated herein by reference in their entirety.

### TECHNICAL FIELD

The invention relates to compounds useful in treating conditions associated with calcium channel function, and particularly conditions associated with T-type calcium channel activity. More specifically, the invention concerns compounds containing amide derivatives and also possessing aromatic functionality that are useful in treatment of conditions such as cardiovascular disease, epilepsy and pain.

### BACKGROUND ART

The entry of calcium into cells through voltage-gated calcium channels mediates a wide variety of cellular and physiological responses, including excitation-contraction coupling, hormone secretion and gene expression (Miller, R. J., Science (1987) 235:46-52; Augustine, G. J. et al., Annu Rev Neurosci (1987) 10: 633-693). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter and calcium channels, which also affects neurite outgrowth and growth cone migration in developing neurons.

Calcium channels mediate a variety of normal physiological functions, and are also implicated in a number of human disorders. Examples of calcium-mediated human disorders include but are not limited to congenital migraine, cerebellar ataxia, angina, epilepsy, hypertension, ischemia, and some arrhythmias. The clinical treatment of some of these disorders has been aided by the development of therapeutic calcium channel antagonists (e.g., dihydropyridines, phenylalkyl amines, and benzothiazapines all target L-type calcium channels) (Janis, R. J. & Triggle, D. J., In Calcium Channels: Their Properties, Functions, Regulation and Clinical Relevance (1991) CRC Press, London).

Native calcium channels have been classified by their electrophysiological and pharmacological properties into T-, L-, N-, P/Q- and R-types (reviewed in Catterall, W., Annu Rev Cell Dev Biol (2000) 16: 521-555; Huguenard, J. R., Annu Rev Physiol (1996) 58: 329-348). T-type (or low voltage-activated) channels describe a broad class of molecules that transiently activate at negative potentials and are highly sensitive to changes in resting potential.

The L-, N- and P/Q-type channels activate at more positive potentials (high voltage-activated) and display diverse kinetics and voltage-dependent properties (Catterall (2000); Huguenard (1996)). T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed α_{1G}, α_{1H}, and α_{1I}.

T-type calcium channels are involved in various medical conditions. In mice lacking the gene expressing the α_{1G} subunit, resistance to absence seizures was observed (Kim, C. et al., Mol Cell Neurosci (2001) 18(2): 235-245). Other studies have also implicated the α_{1H} subunit in the development of epilepsy (Su, H. et al., J Neurosci (2002) 22: 3645-3655). There is strong evidence that some existing anticonvulsant drugs, such as ethosuximide, function through the blockade of T-type channels (Gomora, J. C. et al., Mol Pharmacol (2001) 60: 1121-1132).

Low voltage-activated calcium channels are highly expressed in tissues of the cardiovascular system. Mibefradil, a calcium channel blocker 10-30-fold selective for T-type over L-type channels, was approved for use in hypertension and angina. It was withdrawn from the market shortly after launch due to interactions with other drugs (Heady, T. N., et al., Jpn J. Pharmacol. (2001) 85:339-350).

Growing evidence suggests T-type calcium channels may also be involved in pain (see for example: US Patent Application No. 2003/086980; PCT Patent Application Nos. WO 03/007953 and WO 04/000311). Both mibefradil and ethosuximide have shown anti-hyperalgesic activity in the spinal nerve ligation model of neuropathic pain in rats (Dogrul, A., et al., Pain (2003) 105:159-168). In addition to cardiovascular disease, epilepsy (see also US Patent Application No. 2006/025397), and chronic and acute pain, T-type calcium channels have been implicated in diabetes (US Patent Application No. 2003/125269), certain types of cancer such as prostate cancer (PCT Patent Application Nos. WO 05/086971 and WO 05/77082), sleep disorders (US Patent Application No. 2006/003985), Parkinson's disease (US Patent Application No. 2003/087799); psychosis such as schizophrenia (US Patent Application No. 2003/087799), overactive bladder (Sui, G.-P., et al., British Journal of Urology International (2007) 99(2): 436-441; see also US 2004/197825) and male birth control.

All patents, patent applications and publications are herein incorporated by reference in their entirety.

### DISCLOSURE OF THE INVENTION

The invention relates to compounds useful in treating conditions modulated by calcium channel activity and in particular conditions mediated by T-type channel activity. The compounds of the invention are diamide compounds with structural features that enhance the calcium channel blocking activity of the compounds. Thus, in one aspect, the invention is directed to a method of treating conditions mediated by calcium channel activity by administering to patients in need of such treatment at least one compound of formula (1):

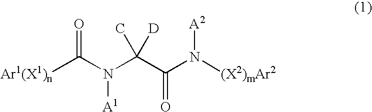

or a pharmaceutically acceptable salt or conjugate thereof, wherein

each X¹ and X² is independently an optionally substituted alkylene (1-3C), alkenylene (2-3C), alkynylene (2-3C), heteroalkylene (2-3C), heteroalkenylene (2-3C), or heteroalkynylene (2-3C);

Ar¹ is an optionally substituted phenyl ring;

Ar² is an optionally substituted aromatic (6-10 membered) or heteroaromatic (5-10 membered) ring;

each A¹ and A² are independently H or methyl;

C is an optionally substituted alkylene (1-3C), alkenylene (2-3C), alkynylene (2-3C), heteroalkylene (2-3C), heteroalkenylene (2-3C), heteroalkynylene (2-3C), aromatic (6-membered) or heteroaromatic (5-10 membered) ring;

D is H, or an optionally substituted alkylene (1-3C), alkenylene (2-3C), alkynylene (2-3C), heteroalkylene (2-3C), heteroalkenylene (2-3C), heteroalkynylene (2-3C),

wherein either C and A¹ or C and D may optionally together form an optionally substituted 3-6 membered cyclic or heterocyclic ring;

n and m are independently 0 or 1; and

wherein the optional substituents on each Ar¹, Ar², X¹, X², C and D are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-3C), alkenyl (2-3C), alkynyl (2-3C), heteroalkyl (2-3C) heteroalkenyl (2-3), and heteroalkynyl (2-3C); or the optional substituents may be one or more optionally substituted groups selected from alkyl (1-3C), alkenyl (2-3C), alkynyl (2-3C), heteroalkyl (2-3C), heteroalkenyl (2-3C), or heteroalkynyl (2-3C); and wherein the optional substituent on C and D may further be selected from ═O and ═NOR′;

and wherein optional substituents on a cyclic or heterocyclic ring formed with C and one of A¹ and D may independently be selected from ═O, ═NOR′, halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-8C), alkenyl (2-8C), alkynyl (2-8C), heteroalkyl (2-8C) heteroalkenyl (2-8C), and heteroalkynyl (2-8C); or the optional substituents may be one or more optionally substituted groups selected from alkyl (1-8C), alkenyl (2-8C), alkynyl (2-8C), heteroalkyl (2-8C), heteroalkenyl (2-8C), heteroalkynyl (2-8C), aromatic (6-10 membered) or heteroaromatic (6-10 membered).

The invention is also directed to the use of compounds of formula (1) for the preparation of medicaments for the treatment of conditions requiring modulation of calcium channel activity, and in particular T-type calcium channel activity. In another aspect, the invention is directed to pharmaceutical compositions containing compounds of formula (1) and to the use of these compositions for treating conditions requiring modulation of calcium channel activity, and particularly T-type calcium channel activity. The invention is also directed to compounds of formula (1) useful to modulate calcium channel activity, particularly T-type channel activity, wherein the definition of such compound is as above with the additional proviso that the compound is not (E)-4-(3-(3-bromo-4-fluorophenyl)acrylamido)-N-(2-(5-fluoro-1H-indol-3-yl)ethyl)piperidine-4-carboxamide.

### DETAILED DESCRIPTION

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-8C (alkyl) or 2-8C (alkenyl or alkynyl). In some embodiments, they contain 1-6C, 1-4C, 1-3C or 1-2C (alkyl); or 2-6C, 2-4C or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In preferred embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In preferred embodiments, the heteroatom is O or N. For greater certainty, to the extent that alkyl is defined as 1-6C, then the corresponding heteroalkyl contains 2-6 C, N, O, or S atoms such that the heteroalkyl contains at least one C atom and at least one heteroatom. Similarly, when alkyl is defined as 1-6C or 1-4C, the heteroform would be 2-6C or 2-4C respectively, wherein one C is replaced by O, N or S. Accordingly, when alkenyl or alkynyl is defined as 2-6C (or 2-4C), then the corresponding heteroform would also contain 2-6 C, N, O, or S atoms (or 2-4) since the heteroalkenyl or heteroalkynyl contains at least one carbon atom and at least one heteroatom. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂), OR, (CH₂), COR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the group contains at least one C and the size of the substituent is consistent with the definition of alkyl, alkenyl and alkynyl.

As used herein, the terms “alkylene,” “alkenylene” and “alkynylene” refers to divalent groups having a specified size, typically 1-2C, 1-3C, 1-4C, 1-6C or 1-8C for the saturated groups and 2-3C, 2-4C, 2-6C or 2-8C for the unsaturated groups. They include straight-chain, branched-chain and cyclic forms as well as combinations of these, containing only C and H when unsubstituted. Because they are divalent, they can link together two parts of a molecule, as exemplified by X in formula (1). Examples include methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

Heteroalkylene, heteroalkenylene and heteroalkynylene are similarly defined as divalent groups having a specified size, typically 2-3C, 2-4C, 2-6C or 2-8C for the saturated groups and 2-3C, 2-4C, 2-6C or 2-8C for the unsaturated groups. They include straight chain, branched chain and cyclic groups as well as combinations of these, and they further contain at least one carbon atom but also contain one or more O, S or N heteroatoms or combinations thereof within the backbone residue, whereby each heteroatom in the heteroalkylene, heteroalkenylene or heteroalkynylene group replaces one carbon atom of the alkylene, alkenylene or alkynylene group to which the heteroform corresponds. As is understood in the art, these heteroforms do not contain more than three contiguous heteroatoms.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, 2-, 3- or 4-pyridyl, indolyl, 2- or 4-pyrimidyl, pyridazinyl, benzothiazolyl or benzimidazolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C, 1-6C or more particularly 1-4C or 1-3C when saturated or 2-8C, 2-6C, 2-4C or 2-3C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′, or NR′SO₂R′, wherein each R′ is independently H or an optionally substituted group selected from alkyl, alkenyl, alkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group, are typically selected from the same list of substituents on aromatic or heteroaromatic groups and may further be selected from ═O and ═NOR′ where R′ is similarly defined.

Halo may be any halogen atom, especially F, Cl, Br, or I, and more particularly it is fluoro, chloro or bromo.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

Ar1 is an optionally substituted phenyl ring. In many embodiments, Ar¹ is unsubstituted or substituted by halo, methyl or CF₃. Ar² is an optionally substituted aromatic (6-10 membered) or heteroaromatic (5-10 membered) ring. In many embodiments Ar² is phenyl or indole. In many embodiments, Ar² is unsubstituted or substituted by halo, methyl, CF₃ or phenoxy.

X¹ and X² may independently be an optionally substituted alkylene, alkenylene, alkynylene, heteroalkylene, heteroalkenylene, or heteroalkynylene (all as defined above). In a more particular embodiment, X¹ and X² may independently be an optionally substituted 1-2C alkylene, and more particularly an optionally substituted ethylene or an optionally substituted ethenylene. In an even more particular embodiment, X¹ is an optionally substituted ethenylene and X² is an optionally substituted ethylene.

C and D are independently an optionally substituted alkyl, alkenyl, alkynyl, heteroalkyl, heteroalkenyl, heteroalkynyl, aryl or heteroaryl as defined above. A¹ and A² are independently H or methyl. In particular embodiments, either C and D or C and A¹ form a three to six membered optionally substituted cyclic or heterocyclic ring. For example, in many embodiments, C and D together form an optionally substituted cyclopropyl, cycloheptyl, cyclohexyl, or piperidyl.

Optional substituents on such ring systems include ═O, ═NOR′, halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl, alkenyl, alkynyl, heteroalkyl, heteroalkenyl, and heteroalkynyl; or the optional substituents may be one or more optionally substituted groups selected from alkyl, alkenyl, alkynyl, heteroalkyl, heteroalkenyl, or heteroalkynyl, aromatic or heteroaromatic ring, or cyclic or heterocyclic ring. More particular examples of such optional substituents include: COCH₃, OH, CH₂CH₂OH, CH₂OH, (CH₂)₂OCH₃, NH(CH₂)₂OCH₃, O(CH₂)₂OCH₃, CH₃, COOCH₃, COCH₂NH₂, CH₂CONH₂, CO(CH₂)₂OCH₃, CONHCH₂CH₃, COCF₃, CONH₂, C(NH)NH₂, CH₂CONH₂, COCH₂NH₂, CH₂CONH₂, COC(OH)(CH₃)₂, COCH₂NH₂, CH₂C(OH)(CH₃)₂, SO₂CH₃, ═NOCH₂CH₃, aromatic (6 membered) or heteroaromatic (5-6 membered) ring, or cyclic or heterocyclic (3-6 membered) ring.

In some preferred embodiments, two or more of the particularly described groups are combined into one compound: it is often suitable to combine one of the specified embodiments of one feature as described above with a specified embodiment or embodiments of one or more other features as described above. For example, a specified embodiment includes C and D forming a piperidyl ring, and another specified embodiment has Ar² as an optionally substituted indolyl group. Thus one preferred embodiment combines both of these features together, i.e., a piperidyl ring in combination with an optionally substituted indolyl. In some specific embodiments, n is 0 and in others n is 1. Thus additional preferred embodiments include n=0 in combination with any of the preferred combinations set forth above; other preferred combinations include n=1 in combination with any of the preferred combinations set forth above.

The compounds of the invention may have ionizable groups so as to be capable of preparation as salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed. It expressly includes both the cis and trans isomers of the cyclopropane rings shown in Formula (1) and (2), although in some embodiments, the trans cyclopropanes are preferred.

Compounds of formula (1) are also useful for the manufacture of a medicament useful to treat conditions characterized by undesired T-type calcium channel activities.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to the compounds of formula (1) when modified so as to be included in a conjugate of this type.

### MODES OF CARRYING OUT THE INVENTION

The compounds of formula (1) are useful in the methods of the invention and exert their desirable effects through their ability to modulate the activity of calcium channels, particularly the activity of T-type calcium channels. This makes them useful for treatment of certain conditions where modulation of T-type calcium channels is desired, including: cardiovascular disease; epilepsy; diabetes; certain types of cancer such as prostate cancer; chronic and acute pain; sleep disorders; Parkinson's disease; psychosis such as schizophrenia; overactive bladder and male birth control.

Cardiovascular disease as used herein includes but is not limited to hypertension, pulmonary hypertension, arrhythmia (such as atrial fibrillation and ventricular fibrillation), congestive heart failure, and angina pectoris.

Epilepsy as used herein includes but is not limited to partial seizures such as temporal lobe epilepsy, absence seizures, generalized seizures, and tonic/clonic seizures.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

For greater certainty, in treating osteoarthritic pain, joint mobility will also improve as the underlying chronic pain is reduced. Thus, use of compounds of the present invention to treat osteoarthritic pain inherently includes use of such compounds to improve joint mobility in patients suffering from osteoarthritis.

It is known that calcium channel activity is involved in a multiplicity of disorders, and particular types of channels are associated with particular conditions. The association of T-type channels in conditions associated with neural transmission would indicate that compounds of the invention which target T-type receptors are most useful in these conditions. Many of the members of the genus of compounds of formula (1) exhibit high affinity for T-type channels. Thus, as described below, they are screened for their ability to interact with T-type channels as an initial indication of desirable function. It is particularly desirable that the compounds exhibit IC₅₀ values of <1 μM. The IC₅₀ is the concentration which inhibits 50% of the calcium, barium or other permeant divalent cation flux at a particular applied potential.

In order to be maximally useful in treatment, it is also helpful to assess the side reactions which might occur. Thus, in addition to being able to modulate a particular calcium channel, it is desirable that the compound has very low activity with respect to the hERG K⁺ channel which is expressed in the heart. Compounds that block this channel with high potency may cause reactions which are fatal. Thus, for a compound that modulates the calcium channel, it should also be shown that the hERG K⁺ channel is not inhibited. Similarly, it would be undesirable for the compound to inhibit cytochrome p450 since this enzyme is required for drug detoxification. Finally, the compound will be evaluated for calcium ion channel type specificity by comparing its activity among the various types of calcium channels, and specificity for one particular channel type is preferred. The compounds which progress through these tests successfully are then examined in animal models as actual drug candidates.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Typical assays are described hereinbelow in Example 17.

Libraries and Screening

The compounds of the invention can be synthesized individually using methods known in the art per se, or as members of a combinatorial library.

Synthesis of combinatorial libraries is now commonplace in the art. Suitable descriptions of such syntheses are found, for example, in Wentworth, Jr., P., et al., Current Opinion in Biol. (1993) 9:109-115; Salemme, F. R., et al., Structure (1997) 5:319-324. The libraries contain compounds with various substituents and various degrees of unsaturation, as well as different chain lengths. The libraries, which contain, as few as 10, but typically several hundred members to several thousand members, may then be screened for compounds which are particularly effective against a specific subtype of calcium channel, e.g., the N-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, K_{d} values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Accordingly, a library of compounds of formula (1) can be used to identify a compound having a desired combination of activities that includes activity against at least one type of calcium channel. For example, the library can be used to identify a compound having a suitable level of activity on T-type calcium channels while having minimal activity on HERG K+ channels.

Utility and Administration

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, therapy; the compounds are formulated in ways consonant with these parameters. A summary of such techniques is found in Remington's Pharmaceutical Sciences, latest edition, Mack Publishing Co., Easton, Pa., incorporated herein by reference.

In general, for use in treatment, the compounds of formula (1) may be used alone, as mixtures of two or more compounds of formula (1) or in combination with other pharmaceuticals. An example of other potential pharmaceuticals to combine with the compounds of formula (1) would include pharmaceuticals for the treatment of the same indication but having a different mechanism of action from T-type calcium channel blocking. For example, in the treatment of pain, a compound of formula (1) may be combined with another pain relief treatment such as an NSAID, or a compound which selectively inhibits COX-2, or an opioid, or an adjuvant analgesic such as an antidepressant. Another example of a potential pharmaceutical to combine with the compounds of formula (1) would include pharmaceuticals for the treatment of different yet associated or related symptoms or indications. Depending on the mode of administration, the compounds will be formulated into suitable compositions to permit facile delivery.

The compounds of the invention may be prepared and used as pharmaceutical compositions comprising an effective amount of at least one compound of formula (1) admixed with a pharmaceutically acceptable carrier or excipient, as is well known in the art. Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the dosage of the compounds of the invention is typically 0.01-15 mg/kg, preferably 0.1-10 mg/kg. However, dosage levels are highly dependent on the nature of the condition, drug efficacy, the condition of the patient, the judgment of the practitioner, and the frequency and mode of administration. Optimization of the dosage for a particular subject is within the ordinary level of skill in the art.

Synthesis of the Invention Compounds

The following reaction schemes and examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

### EXAMPLE 1

### Synthesis of (E)-4-(3-(3-bromo-4-fluorophenyl)acrylamido)-N-(2-(5-fluoro-1H-indol-3-yl)ethyl)piperidine-4-carboxamide (Compound 1)

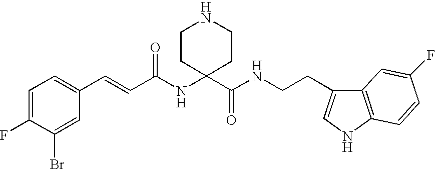

### A. Synthesis of tert-butyl 4-(((9H-fluoren-9-yl)methoxy)carbonylamino)-4-(2-(5-fluoro-1H-indol-3-yl)ethylcarbamoyl)piperidine-1-carboxylate

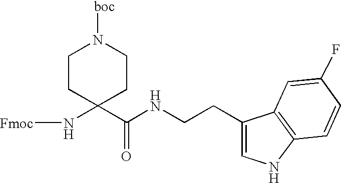

To a solution of 1-N-BOC-4-N-Fmoc-amino-4-carboxylic-piperidine (4.2 g, 6.43 mmol), 5-fluorotryptamine hydrochloride (1.38 g, 6.43 mmol), diisopropylethylamine (DIPEA) (1.83 g, 14.15 mmol) in DMF (35 mL) was added O-(7-azabenzotriazole-1yl)-N,N,N′,N′-tetramethyluronium hexafluorophosphate (HATU) (2.44 g, 6.43 mmol). The solution was stirred for 2 hours at room temperature and concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography 2:8 EtOAc-DCM to give desired compound (4.0 g, 93%) as white foam.

### B. Synthesis of (tert-butyl 4-amino-4-(2-(5-fluoro-1H-indol-3-yl)ethylcarbamoyl)piperidine-1-carboxylate

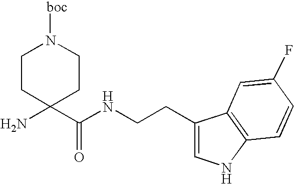

Tert-butyl 4-(((9H-fluoren-9-yl)methoxy)carbonylamino)-4-(2-(5-fluoro-1H-indol-3-yl)ethylcarbamoyl)piperidine-1-carboxylate (4.0 g, 6.4 mmol) was dissolved in the mixture of CH₂Cl₂ (30 mL) and 1,8-diazabicyclo[5.4,0]undec-7-ene (DBU) (1.2 g, 7.7 mmol). The mixture was stirred at room temperature for 1 hour and concentrated in vacuo. The crude material was purified by flash column chromatography on silica gel using 3:3:94 NH4OH/MeOH/DCM to give t-butyl 4-amino-4-(2-(5-fluoro-1H-indol-3-yl)ethylcarbamoyl)piperidine-1-carboxylate (1.84 g, 71%) as white foam.

### C. Synthesis of (E)-tert-butyl4-(3-(3-bromo-4-fluorophenyl)acrylamido)-4-(2-(5-fluoro-1H-indol-3-yl)ethylcarbamoyl)piperidine-1-carboxylate

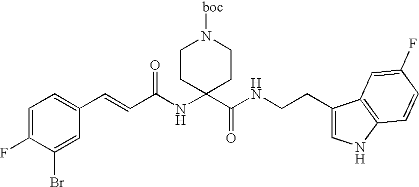

To a solution of t-butyl 4-amino-4-(2-(5-fluoro-1H-indol-3-yl)ethylcarbamoyl)piperidine-1-carboxylate (1.84 g, 4.47 mmol), 4-fluoro-3-bromo-trans-cinnamic acid (1.3 g, 4.57 mmol), diisopropylethylamine (DIPEA) (1.30 mL, 10.06 mmol) in DMF (20 mL) was added O-(7-azabenzotriazole-1yl)-N,N,N′,N′-tetramethyluronium hexafluorophosphate (HATU) (1.74 g, 4.57 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography to give desired compound (2.63 g, 91%) as white foam.

### D. Synthesis of (E)-4-(3-(3-bromo-4-fluorophenyl)acrylamido)-N-(2-(5-fluoro-1H-indol-3-yl)ethyl)piperidine-4-carboxamide

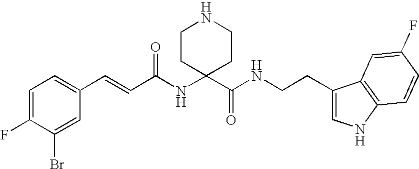

(E)-Tert-butyl-4-(3-(3-bromo-4-fluorophenyl)acrylamido)-4-(2-(5-fluoro-1H-indol-3-yl)ethylcarbamoyl)piperidine-1-carboxylate (2.68 g, 4.17 mmol) was dissolved in CH₂Cl₂ (20 mL) and trifluoroacetic acid (TFA) (5 mL) was added. The mixture was stirred at room temperature for 1 hour. The resulting mixture was neutralized with mixture of saturated aqueous NaHCO₃ (45 mL) and 4N NaOH (15 mL) and the aqueous extracted with CH₂Cl₂. The combined extracts were dried (Na₂SO₄), filtered and concentrated in vacuo. The crude material was used in the next reaction as is or was purified by flash column chromatography on silica gel using 3:3:94 NH₄OH/MeOH/DCM to give (E)-4-(3-(3-bromo-4-fluorophenyl)acrylamido)-N-(2-(5-fluoro-1H-indol-3-yl)ethyl)piperidine-4-carboxamide (1.9 g, 86%) as a white solid.

### EXAMPLE 2

Additional compound nos. 2-10 were prepared according to the general method of Example 1 and the structures for these compounds are shown in Table 3 below. Where applicable, the following intermediates were also synthesized as follows:

### A. Synthesis of 2-(5-fluoro-1H-indol-3-yl)-N-methylethanamine (Intermediate in Synthesis of Compound 4)

### A(i) Synthesis of N-(2-(5-fluoro-1H-indol-3-yl)ethyl)-3,3-dimethylbutanamide

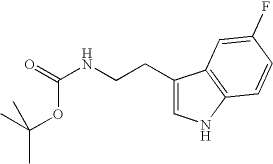

To a solution of 5-fluorotryptamine hydrochloride (510 mg, 2.38 mmol), 2 N NaOH (2.6 mL, 5.2 mmol) in t-BuOH (10 mL) was added BOC₂O (578 mg, 2.65 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo to give N-(2-(5-fluoro-1H-indol-3-yl)ethyl)-3,3-dimethylbutanamide (450 mg, 61%) as white foam.

### A(ii) Synthesis of 2-(5-fluoro-1H-indol-3-yl)-N-methylethanamine

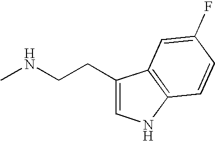

To a solution of N-(2-(5-fluoro-1H-indol-3-yl)ethyl)-3,3-dimethylbutanamide (420 mg, 1.5 mmol) in THF (15 mL) was added LAH powder (288 mg, 7.5 mmol) and the resulting mixture was continued to reflux for 4 hours, and cooled to room temperature. Water (0.3 mL) was slowly added to the reaction mixture followed by 4 N NaOH (0.9 mL, 3.6 mmol) and water (0.3 mL). The resulting suspension was continued to stir for overnight and a white solid was filtered off, and then washed with EtOAC several times. Concentration of the resulting organic gave 2-(5-fluoro-1H-indol-3-yl)-N-methylethanamine (280 mg, 97%) as oil.

### B. Synthesis of (Z)-3-(3-bromo-4-fluorophenyl)-3-fluoroacrylic acid (Intermediate in synthesis of Compound 5)

### B (i) Synthesis of (E)-3-(3-bromo-4-fluorophenyl)-prop-2-en-1-ol

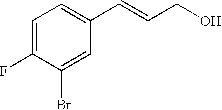

To a solution of (E)-methyl 3-(3-bromo-4-fluorophenyl)acrylate (980 mg, 3.78 mmol) in THF (10 mL) was added DIBAL (1 M in hexane, 9 mL, 9 mmol) at 0° C. The solution was stirred for 3 hour at 0° C., and then quenched with half sat. Na+/K+ tartrate solution. The mixture was exacted with EtOAc, the resulting organic was washed consecutively with saturated aqueous NaHCO₃ and brine. The organic layer was dried (Na₂SO₄), filtered and concentrated in vacuo. The crude material was purified by chromatography on silica gel using 3:7 EtOAc-hexane to give desired (E)-3-(3-bromo-4-fluorophenyl)prop-2-en-1-ol (850 mg, 97%) as white foam.

### B(ii) Synthesis of 2-bromo-3-(3-bromo-4-fluorophenyl)-3-fluoropropan-1-ol

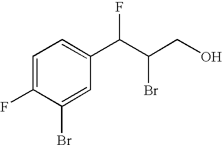

To a solution (E)-3-(3-bromo-4-fluorophenyl)prop-2-en-1-ol (850 mg, 3.28 mmol), in CH₂Cl₂ (55 mL) was slowly added Et₃N (2.1 mL, 12.9 mmol). After 10 min. N-bromosuccinimide (NBS) (1.5 g, 8.4 mmol) was added in two portions. The reaction then was stirred for 5 hours at room temperature and then quenched with sat. NaHCO₃. The mixture was extracted with EtOAc, the resulting organic was washed consecutively with saturated aqueous NaHCO₃ and brine. The organic layer was dried (Na₂SO₄), filtered and concentrated in vacuo. The crude material was purified by chromatography on silica gel using 3:7 EtOAc-hexane to give the desired compound 2-bromo-3-(3-bromo-4-fluorophenyl)-3-fluoropropan-1-ol (1 g, 93%) as a white solid.

### B(iii) Synthesis of (Z)-3-(3-bromo-4-fluorophenyl)-3-fluoroprop-2-en-1-ol

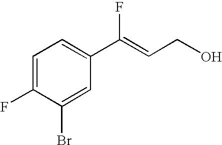

To a solution of 2-bromo-3-(3-bromo-4-fluorophenyl)-3-fluoropropan-1-ol (1 g, 3.03 mmol) in DMSO (100 mL) was added Et₃N (10 mL). The reaction was stirred for 20 hours at 110° C. and cooled to room temperature, the mixture was then partitioned between a mixture of brine (800 mL) and water (800 mL) and extracted with 4×100 CH₂Cl₂. The organic layer was washed with 1 N HCl solution (200 mL), brine and dried over Na₂SO₄. Evaporation of organic solvent and flash chromatography on silica gel using 3:7 EtOAc-hexane gave desired compound (Z)-3-(3-bromo-4-fluorophenyl)-3-fluoroprop-2-en-1-ol (510 mg, 67%) as a white solid.

### B(iv) Synthesis of (Z)-3-(3-bromo-4-fluorophenyl)-3-fluoroacrylaldehyde

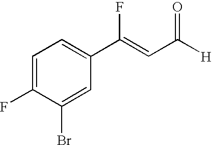

To a solution of (Z)-3-(3-bromo-4-fluorophenyl)-3-fluoroprop-2-en-1-ol (510 mg, 2.04 mmol) in CH₂Cl₂ (20 mL) was added activated MnO₂ (85%, 1.4 g, 14 mmol) and mixture was stirred for 18 hours at room temperature. The reaction mixture was filtered though a short column packed with celite to remove MnO₂. Evaporation of organic solvent and flash chromatography on silica gel using 2:8 EtOAc-hexane gave desired compound (Z)-3-(3-bromo-4-fluorophenyl)-3-fluoroacrylaldehyde (230 mg, 45%) as a white solid.

### B(v) Synthesis of (Z)-3-(3-bromo-4-fluorophenyl)-3-fluoroacrylic acid

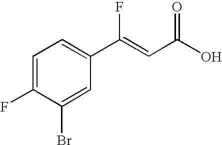

To a solution of (Z)-3-(3-bromo-4-fluorophenyl)-3-fluoroacrylaldehyde (80 mg, 0.32 mmol), 2-methyl-2-butene (2 M in THF, 1.28 mL, 2.56 mmol) in t-BuOH (10 mL) was added solution of NaClO2 (80%, 289 mg, 2.56 mmol) and NaH₂PO₄ (275 mg, 2.56 mmol) in water (3 mL). The solution was stirred at room temperature for 1 hour, quenched with mixture of aq HCl solution (10%, 5 mL) and brine (10 mL). The mixture was then exacted with EtOAc, the resulting organic was washed consecutively with saturated aqueous NaHCO₃ and brine. The organic layer was dried (Na₂SO₄), filtered and concentrated in vacuo. The crude material was purified by radial chromatography on silica gel using 10:90 MeOH/DCM to give desired compound (Z)-3-(3-bromo-4-fluorophenyl)-3-fluoroacrylic acid (70 mg, 87%) as white foam.

### EXAMPLE 3

### Synthesis of N-(2-(5-fluoro-1H-indol-3-yl)ethyl)-4-(3-(4-fluorophenyl)propanamido) piperidine-4-carboxamide (Compound 11)

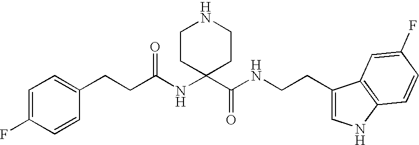

A mixture of (E)-4-(3-(3-bromo-4-fluorophenyl)acrylamido)-N-(2-(5-fluoro-1H-indol-3-yl)ethyl)piperidine-4-carboxamide (0.15 g, 0.28 mmoL, synthesized according to Example 1), palladium hydroxide (10%) (0.10 g), in methanol (10 mL) was stirred under hydrogen at 40 psi. Reaction mixture was diluted with methanol (40 mL) and filtered through celite. The solvent was removed in vacuo to provide crude product. This was purified using flash chromatography 2:6:92 NH₄OH/MeOH/DCM to give N-(2-(5-fluoro-1H-indol-3-yl)ethyl)-4-(3-(4-fluorophenyl)propanamido) piperidine-4-carboxamide (70 mg, 55%) as a white solid.

### EXAMPLE 4

### Synthesis of (E)-1-(3-(3-bromo-4-fluorophenyl)acrylamido)-N-(2-(5-fluoro-1H-indol-3-yl)ethyl)cyclohexanecarboxamide (Compound 12)

### A. Synthesis of tert-butyl 1-(2-(5-fluoro-1H-indol-3-yl)ethylcarbamoyl)cyclohexylcarbamate

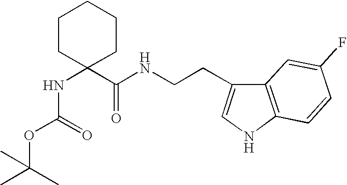

To a solution of 1-(tert-butoxycarbonylamino)cyclohexanecarboxylic acid (210 mg, 0.86 mmol), 5-fluorotryptamine hydrochloride (185 mg, 0.86 mmol), diisopropylethylamine (DIPEA) (0.47 mL, 2.59 mmol) in DMF (5 mL) was added O-(7-azabenzotriazole-1yl)-N,N,N′,N′-tetramethyluronium hexafluorophosphate (HATU) (328 mg, 0.86 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography to give desired compound (280 mg, 80%) as white foam.

### B. Synthesis of 1-amino-N-(2-(5-fluoro-1H-indol-3-yl)ethyl)cyclohexanecarboxamide

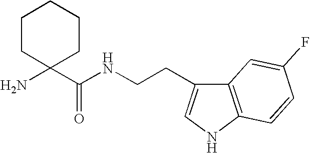

Tert-butyl 1-(2-(5-fluoro-1H-indol-3-yl)ethylcarbamoyl)cyclohexylcarbamate (280 mg, 0.69 mmol) was dissolved in CH₂Cl₂ (4 mL) and trifluoroacetic acid (TFA) (1 mL) was added. The mixture was stirred at room temperature for 1 hour. The resulting mixture was neutralized with mixture of saturated aqueous NaHCO₃ (15 mL) and 4N NaOH (3 mL) and the aqueous extracted with CH₂Cl₂. The combined extracts were dried (Na₂SO₄), filtered and concentrated in vacuo. The crude material was purified by flash column chromatography on silica gel using 3:3:94 NH₃H₂O/MeOH/DCM to give 1-amino-N-(2-(5-fluoro-1H-indol-3-yl)ethyl)cyclohexanecarboxamide (210 mg, 100%) as a white foam.

### C. Synthesis of (E)-1-(3-(3-bromo-4-fluorophenyl)acrylamido)-N-(2-(5-fluoro-1H-indol-3-yl)ethyl)cyclohexanecarboxamide

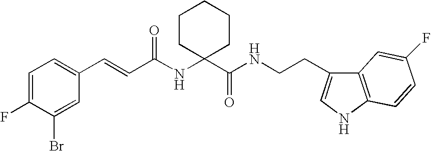

To a solution of 1-amino-N-(2-(5-fluoro-1H-indol-3-yl)ethyl)cyclohexanecarboxamide (210 mg, 0.69 mmol), 3-bromo-4-fluorocinnamic acid (190 mg, 0.78 mmol), diisopropylethylamine (DIPEA) (0.29 mL, 1.56 mmol) in DMF (7 mL) was added O-(7-azabenzotriazole-1yl)-N,N,N′,N′-tetramethyluronium hexafluorophosphate (HATU) (296 mg, 0.78 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-1-(3-(3-bromo-4-fluorophenyl)acrylamido)-N-(2-(5-fluoro-1H-indol-3-yl)ethyl)cyclohexanecarboxamide (300 mg, 81%) as a white foam.

### EXAMPLE 5

### Synthesis of (E)-1-acetyl-4-(3-(3-bromo-4-fluorophenyl)acrylamido)-N-(2-(5-fluoro-1H-indol-3-yl)ethyl)piperidine-4-carboxamide (Compound 21)

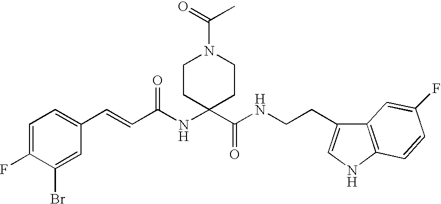

To a solution of (E)-4-(3-(3-bromo-4-fluorophenyl)acrylamido)-N-(2-(5-fluoro-1H-indol-3-yl)ethyl)piperidine-4-carboxamide (110 mg, 0.21 mmol, synthesized according to Example 1) and triethylamine (90 μL, 0.63 mmol) in CH₂Cl₂ (2 mL) was added acetyl chloride (20.6 μL, 0.29 mmol). The solution was stirred at room temperature for 3 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-1-acetyl-4-(3-(3-bromo-4-fluorophenyl)acrylamido)-N-(2-(5-fluoro-1H-indol-3-yl)ethyl)piperidine-4-carboxamide (80 mg, 67%) as a white foam.

### EXAMPLE 6

### Synthesis of (E)-4-(3-(3-bromo-4-fluorophenyl)acrylamido)-N-(2-(5-fluoro-1H-indol-3-yl)ethyl)-1-methylpiperidine-4-carboxamide (Compound 22)

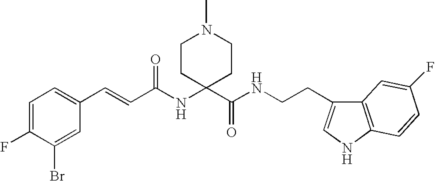

To a solution of (E)-4-(3-(3-bromo-4-fluorophenyl)acrylamido)-N-(2-(5-fluoro-1H-indol-3-yl)ethyl)piperidine-4-carboxamide (120 mg, 0.23 mmol, synthesized according to Example 1) and i-Pr₂NEt (0.1 mL, 0.54 mmol) in THF (2 mL) was added the solution of MeI (32 mg, 0.25 mmol) in THF (1 mL). The solution was stirred at room temperature for 3 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by radial chromatography on silica gel using 3:3:94 NH₄OH/MeOH/DCM to give desired compound (E)-4-(3-(3-bromo-4-fluorophenyl)acrylamido)-N-(2-(5-fluoro-1H-indol-3-yl)ethyl)-1-methylpiperidine-4-carboxamide (15 mg, 11%) as a white foam.

### EXAMPLE 7

### Synthesis of (E)-methyl 4-(3-(3-bromo-4-fluorophenyl)acrylamido)-4-(2-(5-fluoro-1H-indol-3-yl)ethylcarbamoyl)piperidine-1-carboxylate (Compound 23)

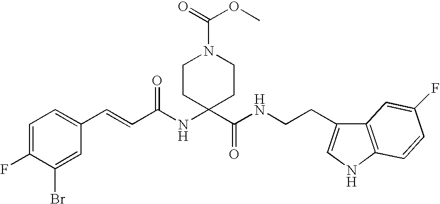

To a solution of (E)-4-(3-(3-bromo-4-fluorophenyl)acrylamido)-N-(2-(5-fluoro-1H-indol-3-yl)ethyl)piperidine-4-carboxamide (100 mg, 0.19 mmol, synthesized according to Example 1) and i-Pr₂NEt (37 μL, 0.37 mmol) in CH₂Cl₂ (2 mL) was added methyl chloroformate (17 μL, 0.22 mmol). The solution was stirred at room temperature for 1 hour, and then quenched with sat. NaHCO₃. The mixture was exacted with EtOAc, the resulting organic was washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-methyl 4-(3-(3-bromo-4-fluorophenyl)acrylamido)-4-(2-(5-fluoro-1H-indol-3-yl)ethylcarbamoyl)piperidine-1-carboxylate (60 mg, 54%) as white foam.

### EXAMPLE 8

### Synthesis of (E)-1-(2-aminoacetyl)-4-(3-(3-bromo-4-fluorophenyl)acrylamido)-N-(2-(5-fluoro-1H-indol-3-yl)ethyl)piperidine-4-carboxamide (Compound 24)

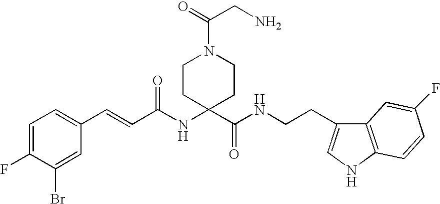

### A. Synthesis of (E)-tert-butyl 2-(4-(3-(3-bromo-4-fluorophenyl)acrylamido)-4-(2-(5-fluoro-1H-indol-3-yl)ethylcarbamoyl))piperidine-1-yl)-2-oxoethylcarbamate

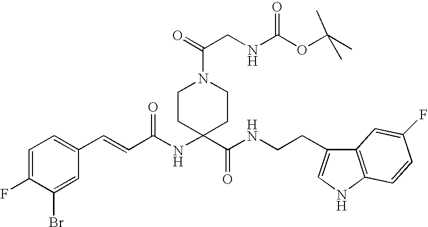

To a solution of (E)-4-(3-(3-bromo-4-fluorophenyl)acrylamido)-N-(2-(5-fluoro-1H-indol-3-yl)ethyl)piperidine-4-carboxamide (130 mg, 0.24 mmol, synthesized according to Example 1), BOC-Gly-OH (43 mg, 0.24 mmol), diisopropylethylamine (DIPEA) (0.10 mL, 0.55 mmol) in DMF (2 mL) was added O-(7-azabenzotriazole-1yl)-N,N,N′,N′-tetramethyluronium hexafluorophosphate (HATU) (93 mg, 0.24 mmol). The solution was stirred at room temperature for 18 hours, and then concentrated in vacuo. The residue was diluted with EtOAc, washed consecutively with saturated aqueous NaHCO₃ and brine. The organic layer was dried (Na₂SO₄), filtered and concentrated in vacuo to give the desired product as crude (200 mg) as a white foam.

### B. Synthesis of (E)-1-(2-aminoacetyl)-4-(3-(3-bromo-4-fluorophenyl)acrylamido)-N-(2-(5-fluoro-1H-indol-3-yl)ethyl)piperidine-4-carboxamide

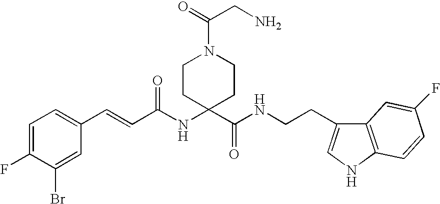

(E)-tert-butyl 2-(4-(3-(3-bromo-4-fluorophenyl)acrylamido)-4-(2-(5-fluoro-1H-indol-3-yl)ethylcarbamoyl))piperidine-1-yl)-2-oxoethylcarbamate (200 mg) was dissolved in CH₂Cl₂ (4 mL) and trifluoroacetic acid (TFA) (1 mL) was added. The mixture was stirred at room temperature for 1 hour. The resulting mixture was neutralized with mixture of saturated aqueous NaHCO₃ (15 mL) and 4N NaOH (3 mL) and the aqueous extracted with CH₂Cl₂. The combined extracts were dried (Na₂SO₄), filtered and concentrated in vacuo. The crude material was purified by radial chromatography on silica gel using 3:3:94 NH₄OH/MeOH/DCM to give (E)-1-(2-aminoacetyl)-4-(3-(3-bromo-4-fluorophenyl)acrylamido)-N-(2-(5-fluoro-1H-indol-3-yl)ethyl)piperidine-4-carboxamide (90 mg, 62% in two steps) as a white foam.

### EXAMPLE 9

### Synthesis of (E)-4-(3-(3-bromo-4-fluorophenyl)acrylamido)-N-(2-(5-fluoro-1H-indol-3-yl)ethyl)-1-(3-methoxypropanol)piperidine-4-carboxamide (Compound 25)

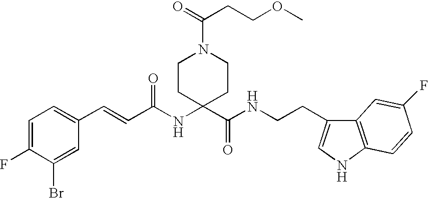

To a solution of (E)-4-(3-(3-bromo-4-fluorophenyl)acrylamido)-N-(2-(5-fluoro-1H-indol-3-yl)ethyl)piperidine-4-carboxamide (100 mg, 0.18 mmol, synthesized according to Example 1), 3-methoxypropionic acid (20 μL, 0.21 mmol), diisopropylethylamine (DIPEA) (77 μL, 0.42 mmol) in DMF (2 mL) was added O-(7-azabenzotriazole-1yl)-N,N,N′,N′-tetramethyluronium hexafluorophosphate (HATU) (68 mg, 0.18 mmol). The solution was stirred at room temperature for 18 hours, and then concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-4-(3-(3-bromo-4-fluorophenyl)acrylamido)-N-(2-(5-fluoro-1H-indol-3-yl)ethyl)-1-(3-methoxypropanol)piperidine-4-carboxamide (70 mg, 63%) as a white foam.

### EXAMPLE 10

### Synthesis of (E)-4-(3-(3-bromo-4-fluorophenyl)acrylamido)-N1-ethyl-N4-(2-(5-fluoro-1H-indol-3-yl)ethyl)piperidine-1,4-dicarboxamide (Compound 26)

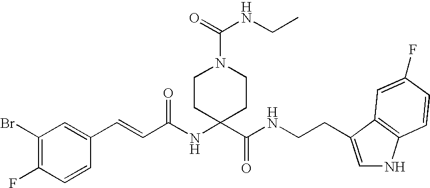

To a suspension of (E)-4-(3-(3-bromo-4-fluorophenyl)acrylamido)-N-(2-(5-fluoro-1H-indol-3-yl)ethyl)piperidine-4-carboxamide (80 mg, 0.15 mmol, synthesized according to Example 1) in CH₂Cl₂ (2 mL) was added ethyl isocyanate (17 μL, 0.21 mmol) followed by 2 drops of DMF to dissolve the solid. The solution was stirred at room temperature for 2 hours, and then quenched with sat. NaHCO₃. The mixture was exacted with EtOAc, the resulting organic was washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give the desired compound (E)-4-(3-(3-bromo-4-fluorophenyl)acrylamido)-N1-ethyl-N4-(2-(5-fluoro-1H-indol-3-yl)ethyl)piperidine-1,4-dicarboxamide (70 mg, 78%) as white foam.

### EXAMPLE 11

### Synthesis of (E)-4-(3-(3-bromo-4-fluorophenyl)acrylamido)-N-(2-(5-fluoro-1H-indol-3-yl)ethyl)-1-(2,2,2-trifluoroacetyl)piperidine-4-carboxamide (Compound 27)

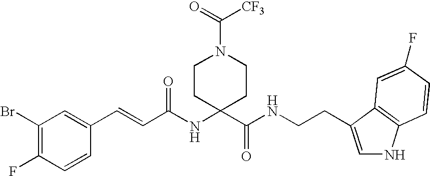

To a solution of (E)-4-(3-(3-bromo-4-fluorophenyl)acrylamido)-N-(2-(5-fluoro-1H-indol-3-yl)ethyl)piperidine-4-carboxamide (120 mg, 0.23 mmol, synthesized according to Example 1) and i-Pr₂NEt (0.1 mL, 0.54 mmol) in CH₂Cl₂ (2 mL) was added trifluoroacetic anhydride (57 mg, 0.27 mmol). The solution was stirred at room temperature for 3 hours, and then quenched with sat. NaHCO₃. The mixture was exacted with EtOAc, the resulting organic was washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-4-(3-(3-bromo-4-fluorophenyl)acrylamido)-N-(2-(5-fluoro-1H-indol-3-yl)ethyl)-1-(2,2,2-trifluoroacetyl)piperidine-4-carboxamide (100 mg, 71%) as a white foam.

### EXAMPLE 12

### Synthesis of (E)-1-(3-(3-bromo-4-fluorophenyl)acrylamido)-N1-(2-(5-fluoro-1H-indol-3-yl)ethyl)cyclohexane-1,4-dicarboxamide (Compound 28)

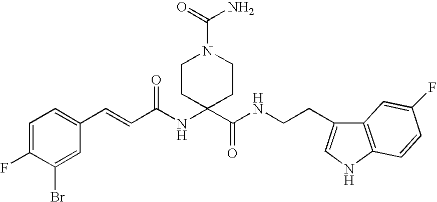

To a solution of (E)-4-(3-(3-bromo-4-fluorophenyl)acrylamido)-N-(2-(5-fluoro-1H-indol-3-yl)ethyl)piperidine-4-carboxamide (100 mg, 0.19 mmol, synthesized according to Example 1) in a mixture of 2-propanol (2 mL) and CH₂Cl₂ (1 mL) was added trimethylsilyl isocyanate (40 μL, 0.28 mmol). The solution was stirred at room temperature for 18 hours, and then quenched with sat. NaHCO₃. The mixture was exacted with EtOAc, the resulting organic was then washed consecutively with saturated aqueous NaHCO₃ and brine. The organic layer was dried (Na₂SO₄), filtered and concentrated in vacuo. The crude material was purified by radial chromatography on silica gel using 1:1:98 NH₄OH/MeOH/DCM to give desired compound (E)-1-(3-(3-bromo-4-fluorophenyl)acrylamido)-N1-(2-(5-fluoro-1H-indol-3-yl)ethyl)cyclohexane-1,4-dicarboxamide (90 mg, 83%) as a white foam.

### EXAMPLE 13

### Synthesis of (E)-(4-(3-(3-bromo-4-fluorophenyl)acrylamido)-1-carbamimidoyl-N-(2-(5-fluoro 1H-indol-3-yl)ethyl)piperidine-4-dicarboxamide hydrochloride (Compound 29)

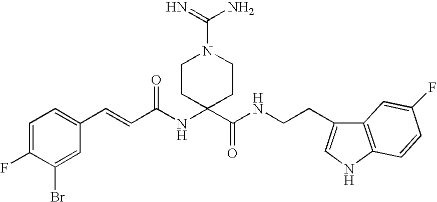

To a solution of (E)-4-(3-(3-bromo-4-fluorophenyl)acrylamido)-N-(2-(5-fluoro-1H-indol-3-yl)ethyl)piperidine-4-carboxamide (100 mg, 0.19 mmol, synthesized according to Example 1) and i-Pr₂NEt (0.17 mL, 0.96 mmol) in DMF (2 mL) was added 1H-pyrazole-carboxamidine hydrochloride (28 mg, 0.19 mmol). The solution was stirred at room temperature for 18 hours, and then quenched with sat. NaHCO₃. The mixture was extracted with EtOAc, the resulting organic was then washed consecutively with saturated aqueous NaHCO₃ and brine. The organic layer was dried (Na₂SO₄), filtered and concentrated in vacuo. The crude material was purified by radial chromatography on silica gel using 3:8:88 NH₄OH/MeOH/DCM to give free amine. The free amine was treated with 2 N HCl solution in diethylether (3 mL) to give desired compound (E)-1-(3-(3-bromo-4-fluorophenyl)acrylamido)-N1-(2-(5-fluoro-1H-indol-3-yl)ethyl)cyclohexane-1,4-dicarboxamide hydrochloride (80 mg, 69%) as a white foam.

### EXAMPLE 14

### Synthesis of (E)-4-(3-(3-bromo-4-fluorophenyl)-N-methyl acrylamido)-N-(2-(5-fluoro-1H-indol-3-yl)ethyl)piperidine-4-carboxamide (Compound 30)

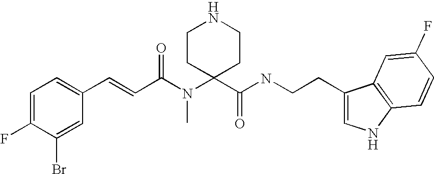

### A. Synthesis of (E)-1-tert-butyl 4-methyl 4-(3-(3-bromo-4-fluorophenyl)acrylamido)piperidine-1,4-dicarboxylate

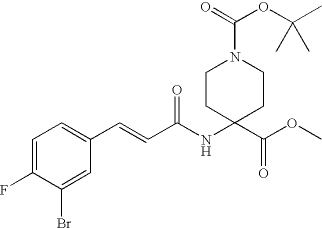

To a solution of 4-N-Boc-amino-piperidine-4-carboxylic acid methyl ester (200 mg, 0.77 mmol), 4-fluoro-3-bromo-trans-cinnamic acid (189 mg, 0.77 mmol), di-isopropylethylamine (DIPEA) (0.281 mL, 1.54 mmol) in DMF (7 mL) was added O-(7-azabenzotriazole-1yl)-N,N,N′,N′-tetramethyluronium hexafluorophosphate (HATU) (290 mg, 0.77 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography using 2:8 EtOAc-DCM to give (E)-1-tert-butyl 4-methyl 4-(3-(3-bromo-4-fluorophenyl)acrylamido)piperidine-1,4-dicarboxylate (350 mg, 93%) as a white foam.

### B. Synthesis of (E)-1-tert-butyl 4-methyl-4-(3-(3-bromo-4-fluorophenyl)-N-methylacrylamido) piperidine-1,4-dicarboxylate

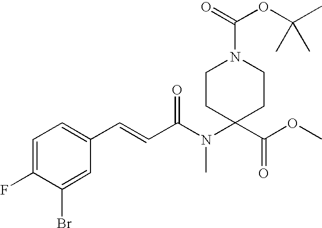

To a solution of (E)-1-tert-butyl 4-methyl 4-(3-(3-bromo-4-fluorophenyl)acrylamido) piperidine-1,4-dicarboxylate (350 mg, 0.72 mmol) in THF (7 mL) was added NaH (60%, 50 mg, 1.25 mmol) at room temperature. The reaction mixture was continued to stir for half hour at r.t. and MeI (0.25 mL, 4.0 mmol) was added. After 18 hours, the reaction mixture was diluted with EtOAc and washed consecutively with saturated aqueous NaHCO₃ and brine. The organic layer was dried (Na₂SO₄), filtered and concentrated in vacuo. The crude material was purified by flash column chromatography using 2:8 EtOAc-DCM to give (E)-1-tert-butyl 4-methyl-4-(3-(3-bromo-4-fluorophenyl)-N-methylacrylamido) piperidine-1,4-dicarboxylate (190 mg, 53%) as a white foam.

### C. Synthesis of (E)-4-(3-(3-bromo-4-fluorophenyl)-N-methylacrylamido)-1-(tert-butoxycarbonyl)piperidine-4-carboxylic acid

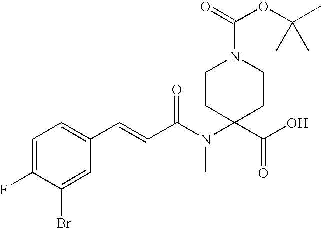

To a solution of (E)-1-tert-butyl 4-methyl-4-(3-(3-bromo-4-fluorophenyl)-N-methylacrylamido) piperidine-1,4-dicarboxylate (190 mg, 0.38) in MeOH (4 mL) was added NaOH solution (4 N, 0.5 mL, 2.00 mmol). The resulting mixture was refluxed for 4 h, cooled to room temperature and concentrated in vacuo. The residue was re-dissolved in water (5 mL) and neutralized with 6 N HCl solution until pH was equal to 4. A white solid was collected, then dried over 24 h in oven under high vacuo to give (E)-4-(3-(3-bromo-4-fluorophenyl)-N-methylacrylamido)-1-(tert-butoxycarbonyl)piperidine-4-carboxylic acid (130 mg) as a white powder.

### D. Synthesis of (E)-tert-butyl 4-(3-(3-bromo-4-fluorophenyl)-N-methylacrylamido)-4-(2-(5-fluoro-1H-indol-3-yl)ethylcarbamoyl)piperidine-1-carboxylate

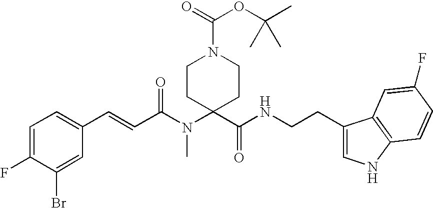

To a solution of (E)-4-(3-(3-bromo-4-fluorophenyl)-N-methylacrylamido)-1-(tert-butoxycarbonyl)piperidine-4-carboxylic acid (130 mg, 0.26 mmol), 5-fluorotryptamine hydrochloride (60 mg, 0.26 mmol), diisopropylethylamine (DIPEA) (0.15 mL, 0.80 mmol) in DMF (3 mL) was added O-(7-azabenzotriazole-1yl)-N,N,N′,N′-tetramethyluronium hexafluorophosphate (HATU) (102 mg, 0.26 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography to give (E)-tert-butyl 4-(3-(3-bromo-4-fluorophenyl)-N-methylacrylamido)-4-(2-(5-fluoro-1H-indol-3-yl)ethylcarbamoyl)piperidine-1-carboxylate (120 mg, 68%) as white foam.

### E. Synthesis of (E)-4-(3-(3-bromo-4-fluorophenyl)-N-methyl acrylamido)-N-(2-(5-fluoro-1H-indol 3-yl)ethyl)piperidine-4-carboxamide

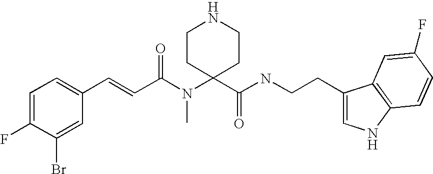

To a solution of (E)-tert-butyl 4-(3-(3-bromo-4-fluorophenyl)-N-methylacrylamido)-4-(2-(5-fluoro-1H-indol-3-yl)ethylcarbamoyl)piperidine-1-carboxylate (120 mg, 0.18 mmol) in CH₂Cl₂ was added TFA (1 mL). The mixture was stirred at room temperature for 1 hour. The resulting mixture was neutralized with mixture of saturated aqueous NaHCO₃ (15 mL) and 4N NaOH (3 mL) and the aqueous extracted with CH₂Cl₂. The combined extracts were dried (Na₂SO₄), filtered and concentrated in vacuo. The crude material was purified by flash column chromatography on silica gel using 3:3:94 NH₄OH/MeOH/DCM to give (E)-4-(3-(3-bromo-4-fluorophenyl)-N-methyl acrylamido)-N-(2-(5-fluoro-1H-indol 3-yl)ethyl)piperidine-4-carboxamide (60 mg, 61%) as a white foam.

### EXAMPLE 15

### Synthesis of (E)-3-(3,4-difluorophenyl)-N-(3-(dimethylamino)-1-(2-(5-fluoro-1H-indol-3-yl)ethylamino)-1-oxopropan-2-yl)acrylamide (Compound 31)

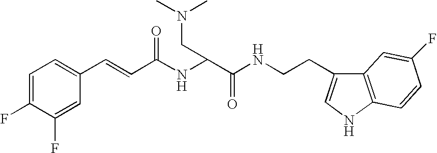

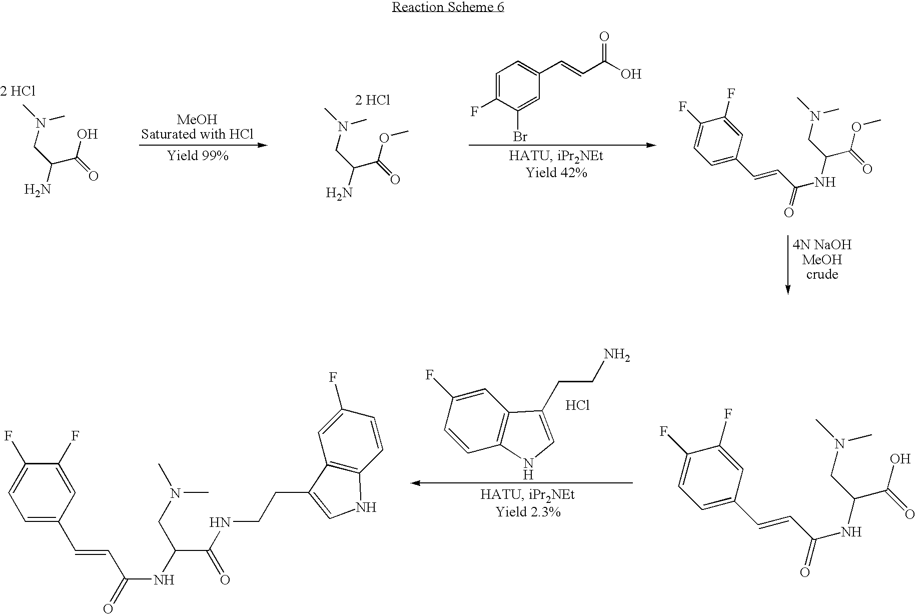

### A. Synthesis of methyl 2-amino-3-(dimethylamino)propanoate

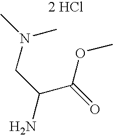

Methanol (15 mL) was saturated with anhydrous hydrogen chloride gas, and to this solution was added 2-amino-3-(dimethylamino)propanoic acid (0.5 g, 2.43 mmoL) and stirred at 20° C. for 18 h. Reaction mixture was then concentrated and triturated with anhydrous diethyl ether to get methyl 2-amino-3-(dimethylamino)propanoate (0.53 g, 99%) as a white solid. MS: 147.2 (M+1).

### B. Synthesis of (E)-methyl 2-(3-(3,4-difluorophenyl)acrylamido)-3-(dimethylamino)propanoate

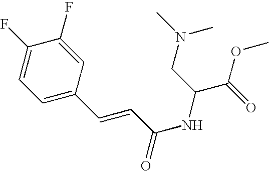

A mixture of methyl 2-amino-3-(dimethylamino)propanoate (1.50 g, 6.85 mmoL), (O-(7-azabenzotriazol-1-YL)-N,N,N′,N′-tetramethyluronium (HATU) (2.60 g, 6.85 mmoL), (E)-3-(3-bromo-4-fluorophenyl)acrylic acid (1.26 g, 6.85 mmoL), and N,N-diisopropylethylamine (iPr2NEt) (5.57 g, 43.06 mmoL) in anhydrous dimethylformamide (15 mL) was stirred at 20° C. for 18 h. The volatile components were then removed in vacuo, the residue was dissolved in ethyl acetate (400 mL), washed with saturated aqueous sodium bicarbonate solution (2×100 mL) followed by saturated aqueous ammonium chloride solution (100 mL) and dried over anhydrous sodium sulfate, and the solvent was removed in vacuo to provide crude product. This was purified using flash chromatography (ethyl acetate:methanol 95:5) to obtain (E)-methyl 2-(3-(3,4-difluorophenyl)acrylamido)-3-(dimethylamino)propanoate (0.9 g, yield 42%) as a light yellow solid. MS: 313.3 (M+1).

### C. Synthesis of E)-2-(3-(3,4-difluorophenyl)acrylamido)-3-(dimethylamino)propanoic acid hydrochloride

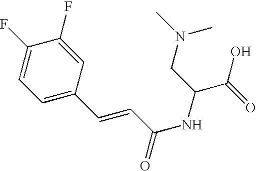

A mixture of (E)-methyl 2-(3-(3,4-difluorophenyl)acrylamido)-3-(dimethylamino)propanoate (0.9 g, 2.89 mmol), and 2N sodium hydroxide (4.8 mL) in methanol (5 mL) was refluxed for 2 days. The reaction mixture was extracted with ethyl acetate (20 mL). The aqueous layer was acidified with 10N HCl to Ph 4.5. Reaction mixture was concentrated and lyophilized. (E)-2-(3-(3,4-difluorophenyl)acrylamido)-3-(dimethylamino)propanoic acid hydrochloride was obtained as crude light yellow solid (1.15 g) and was taken to next step without any further purification. MS: 299.5 (M−1).

### D. Synthesis of (E)-3-(3,4-difluorophenyl)-N-(3-(dimethylamino)-1-(2-(5-fluoro-1H-indol-3-yl)ethylamino)-1-oxopropan-2-yl)acrylamide

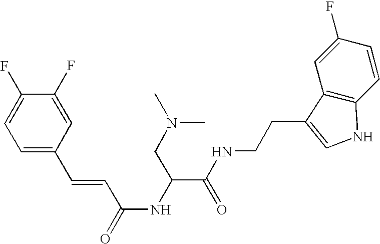

A mixture of (E)-2-(3-(3,4-difluorophenyl)acrylamido)-3-(dimethylamino)propanoic acid hydrochloride (0.87 g, crude), (O-(7-azabenzotriazol-1-YL)-N,N,N′,N′-tetramethyluronium (HATU) (0.38 g, 1.00 mmoL), 5-fluorotryptamine hydrochloride (0.21 g, 1.0 mmoL), and N,N-diisopropylethylamine (iPr2NEt) (0.47 g, 3.63 mmoL) in anhydrous dimethylformamide (5 mL) was stirred at 20° C. for 18 h. The volatile components were then removed in vacuo, the residue was dissolved in ethyl acetate (100 mL), and washed with saturated aqueous sodium bicarbonate solution (10 mL) followed by saturated aqueous sodium chloride solution (10 mL) and then dried over anhydrous sodium sulfate. The solvent was removed in vacuo to provide crude product. This was purified using flash chromatography (ethyl acetate) to obtain (E)-3-(3,4-difluorophenyl)-N-(3-(dimethylamino)-1-(2-(5-fluoro-1H-indol-3-yl)ethylamino)-1-oxopropan-2-yl)acrylamide (0.03 g, yield 2.3% over 2 steps) as a light yellow solid. MS: 459.2 (M+1).

### EXAMPLE 16

### Synthesis of (E)-N-(3,5-bis(trifluoromethyl)phenyl)-4-(3-(4-fluorophenyl)acrylamido) piperidine-4-carboxamide (Compound 32)

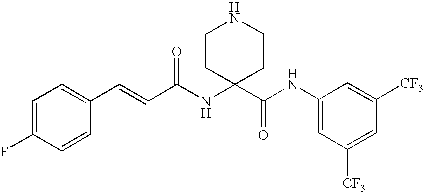

### A. Synthesis of tert-butyl 4-((9H-fluoren-9-yl)methoxy)carbonylamino)-4-(3,5-bis(trifluoromethyl)phenylcarbamoyl)piperidine-1-carboxylate

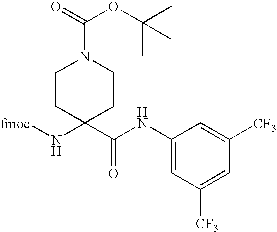

To a solution of 1-N-BOC-4-N-Fmoc-amino-4-carboxylic-piperidine (150 mg, 0.30 mmol), 3,5-bis(trifluoromethyl)aniline (0.15 mL, 0.96 mmol), diisopropylethylamine (DIPEA) (0.12 mL, 0.32 mmol) in DMF (2 mL) was added O-(7-azabenzotriazole-1yl)-N,N,N′,N′-tetramethyluronium hexafluorophosphate (HATU) (120 mg, 0.30 mmol). The solution was stirred in microwave for 0.5 hours at 80° C. and concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography using 2:8 EtOAc-DCM to give desired compound (130 mg, 59%) as a white foam.

### B. Synthesis of tert-butyl 4-amino-4-bis(trifluoromethyl)phenylcarbamoyl)piperidine-1-carboxylate

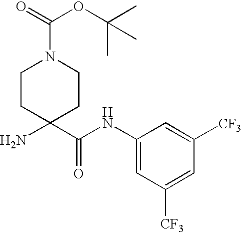

Tert-butyl 4-((9H-fluoren-9-yl)methoxy)carbonylamino)-4-(3,5-bis(trifluoromethyl)phenylcarbamoyl)piperidine-1-carboxylate (130 mg, 0.19 mmol) was dissolved in the mixture of CH₂Cl₂ (4 mL) and 1,8-diazabicyclo[5.4,0]undec-7-ene (DBU) (50 μL, 0.4 mmol). The mixture was stirred at room temperature for 1 hour and concentrated in vacuo. The crude material was purified by flash column chromatography on silica gel using 3:3:94 NH4OH/MeOH/DCM to give butyl 4-amino-4-(3,5)-bis(trifluoromethyl)phenylcarbamoyl)piperidine-1-carboxylate (80 mg, 92%) as a white foam.

### C. Synthesis of (E)-tert-butyl 4-(3,5-bis(trifluoromethyl)phenylcarbamoyl-4-(3-(4-fluorophenyl)acrylamido) piperidine-1-carboxylate

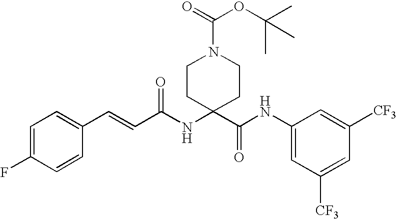

To a solution of bis(trifluoromethyl)phenylcarbamoyl)piperidine-1-carboxylate (80 mg, 0.18 mmol), 4-fluoro-trans-cinnamic acid (29 mg, 0.18 mmol), diisopropylethylamine (DIPEA) (0.10 mL, 0.54 mmol) in DMF (2 mL) was added O-(7-azabenzotriazole-1yl)-N,N,N′,N′-tetramethyluronium hexafluorophosphate (HATU) (67 mg, 0.16 mmol). The solution was stirred at room temperature for 18 hours, concentrated in vacuo. The residue was diluted with EtOAc, washed consecutively with saturated aqueous NaHCO₃ and brine. The organic layer was dried (Na₂SO₄), filtered and concentrated in vacuo. The crude material was purified by flash column chromatography to give desired compound (88 mg, 84%) as a white foam.

### D. Synthesis of (E)-N-(3,5-bis(trifluoromethyl)phenyl)-4-(3-(4-fluorophenyl)acrylamido) piperidine-4-carboxamide

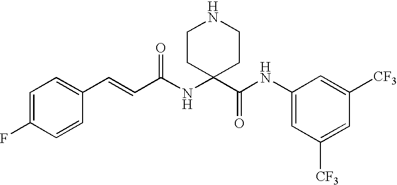

(E)-tert-butyl 4-(3,5-bis(trifluoromethyl)phenylcarbamoyl-4-(3-(4-fluorophenyl)acrylamido) piperidine-1-carboxylate (88 mg, 0.14 mmol) was dissolved in CH₂Cl₂ (4 mL) and trifluoroacetic acid (TFA) (1 mL) was added. The mixture was stirred at room temperature for 1 hour. The resulting mixture was neutralized with mixture of saturated aqueous NaHCO₃ (15 mL) and 4N NaOH (3 mL) and the aqueous extracted with CH₂Cl₂. The combined extracts were dried (Na₂SO₄), filtered and concentrated in vacuo. The crude material was purified by flash column chromatography on silica gel using 3:3:94 NH₄OH/MeOH/DCM to give (E)-N-(3,5-bis(trifluoromethyl)phenyl)-4-(3-(4-fluorophenyl)acrylamido) piperidine-4-carboxamide (37 mg, 52%) as a white foam.

### EXAMPLE 17

Following the general procedures set forth in Examples 1-16, the following compounds listed in Table 1 below were prepared. Mass spectrometry was employed with the final compound and at various stages throughout the synthesis as a confirmation of the identity of the product obtained (M+1). For the mass spectrometric analysis, samples were prepared at an approximate concentration of 1 μg/mL in acetonitrile with 0.1% formic acid. Samples were then manually infused into an Applied Biosystems API3000 triple quadrupole mass spectrometer and scanned in Q1 in the range of 50 to 700 m/z.

TABLE 1

| Cmpd | | | Mass Spec |
|---|---|---|---|
| No. | Name | Structure | (m/z) |
| 1 | (E)-4-(3-(3-bromo-4-fluorophenyl)acrylamido)-N-(2-(5-fluoro-1H-indol-1-yl)ethyl)piperidine-4-carboxamide | 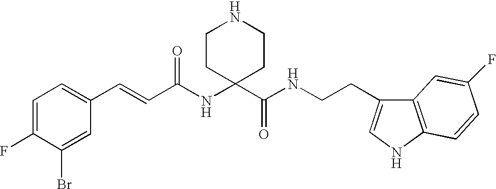 | 531.2 |
| 2 | (E)-N-(2-(1H-indol-3-yl)ethyl)-4-(3-(3-bromo-4-fluorophenyl)acrylamido)piperidine-4-carboxamide | 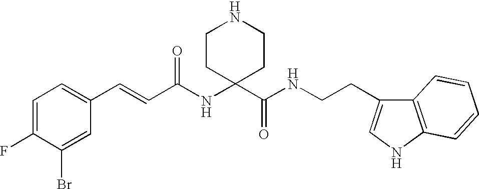 | 513.2 |
| 3 | (E)-4-(3-(3-bromophenyl-acrylamido)-N-(2-(5-fluoro-1H-indol-3-yl)ethyl)piperidine-4-carboxamide | 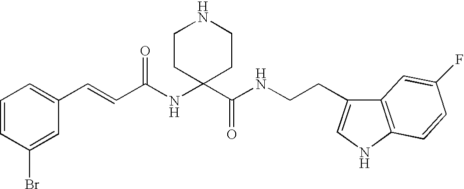 | 513.2 |
| 4 | (E)-4-(3-(3-bromo-4-fluorophenyl)acrylamido)-N-(2-(5-fluoro-1H-indol-3-yl) ethyl)-N-methylpiperidine-4-carboxamide | 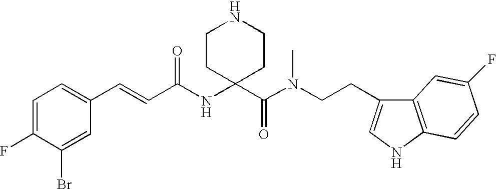 | 545.1 |
| 5 | (Z)-4-(3-(3-bromo-4-fluorophenyl)-3-fluoroacrylamido)-N-(2-(5-fluoro-1H-indol-3-yl)ethyl) piperidine-4-carboxamide | 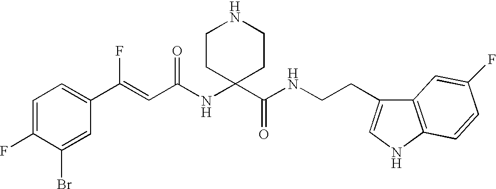 | 549.4 |
| 6 | (E)-4-(3-(3-bromo-4-fluorophenyl)acrylamido)-N-(2,4-difluorobenzyl)piperidine-4-carboxamide | 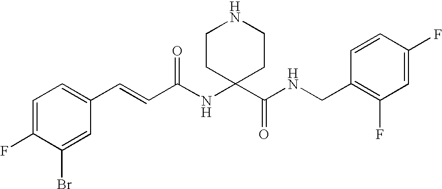 | 496.1 |
| 7 | (E)-4-(3-(3,4-difluorophenyl)acrylamido)-N-(2-(5-fluoro-1H-indol-3-yl)ethyl)piperidine-4-carboxamide | 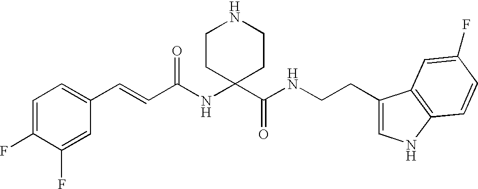 | 471.2 |
| 8 | (E)-N-(2-(5-fluoro-1H-indol-3-yl)ethyl)-4-(3-(4-fluorophenyl)acrylamido)piperidine-4-carboxamide | 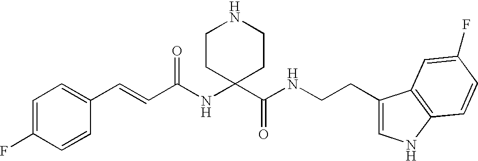 | 453.3 |
| 9 | (E)-4-(3-(3-chloro-4-fluorophenyl)acrylamido)-N-(2-(5-fluoro-1H-indol-3-yl)ethyl) piperidine-4-carboxamide | 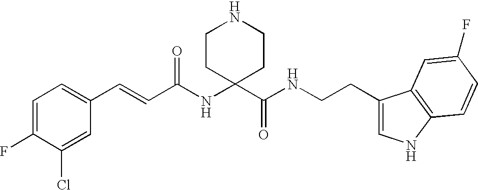 | 487.3 |
| 10 | (E)-4-(3-(5-fluorophenyl) acrylamido)-N-(2-(5-fluoro-1H-indol-3-yl)ethyl)piperidine-4-carboxamide | 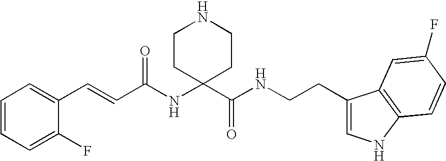 | 453.2 |
| 11 | N-(2-(5-fluoro-1H-indol-3-yl)ethyl)-4-(3-(4-fluorophenyl)propanamido)piperidine-4-carboxamide | 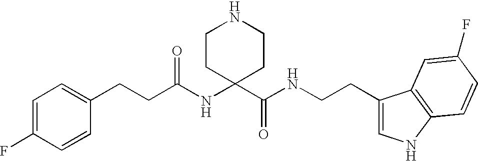 | 455.3 |
| 12 | (E)-1-(3-(3-bromo-4-fluorophenyl)acrylamido)-N-(2-(5-fluoro-1H-indol-3-yl)ethyl)cyclohexanecarboxamide | 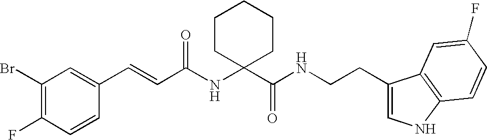 | 530.1 |
| 13 | (E)-1-(3-(3-bromo-4-fluorophenyl)acrylamido)-N-(2-(5-fluoro-1H-indol-3-yl)ethyl)cyclopentanecarboxamide | 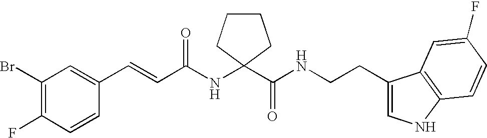 | 516.5 |
| 14 | (E)-1-(3-(3-bromo-4-fluorophenyl)acrylamido)-N-(2-(5-fluoro-1H-indol-3-yl)ethyl)cyclopropanecarboxamide | 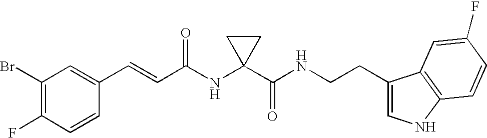 | 488.3 |
| 15 | (E)-3-(3-bromo-4-fluorophenyl)-N-(1-(2-(5-fluoro-1H-indol-3-yl)ethylamino)-2-methyl-1-oxopropan-2-yl)acrylamide | 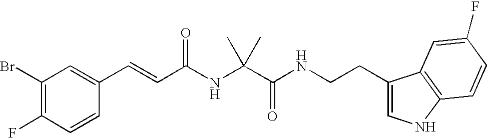 | 490.9 |
| 16 | (E)-4-(3-(3-bromo-4-fluorophenyl)acrylamido)-N-(2-(5-fluoro-1H-indol-3-yl)ethyl)tetrahydro-2H-pyran-4-carboxamide | 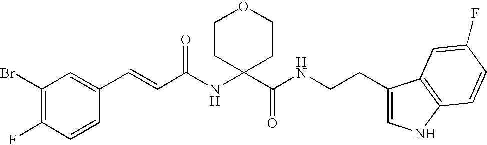 | 532.2 |
| 17 | (S,E)-3-(3-bromo-4-fluorophenyl)-N-(2-(2-(5-fluoro-1H-indol-3-yl)ethylamino)-2-oxo-1-phenylethyl)acrylamide | 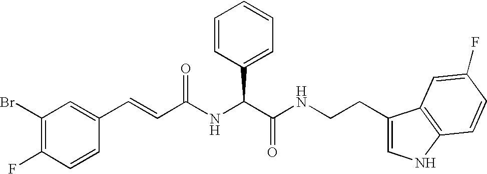 | 538.0 |
| 18 | (R,E)-3-(3-bromo-4-fluorophenyl)-N-(2-(2-(5-fluoro-1H-indol-3-yl)ethylamino)-2-oxo-1-phenylethyl)acrylamide | 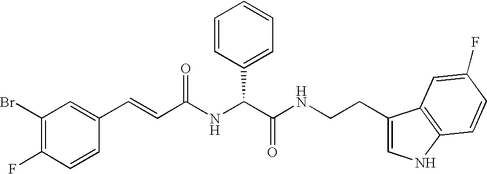 | 538.1 |
| 19 | (R,E)-1-(3-(3-bromo-4-fluorophenyl)acryloyl)-N-(2-(5-fluoro-1H-indol-3-yl)ethyl)pyrrolidine-2-carboxamide | 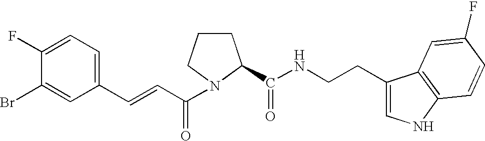 | 503.9 |
| 20 | (R,E)-1-(3-(3-bromo-4-fluorophenyl)acryloyl)-N-(2-(5-fluoro-1H-indol-3-yl)ethyl)pyrrolidine-2-carboxamide | 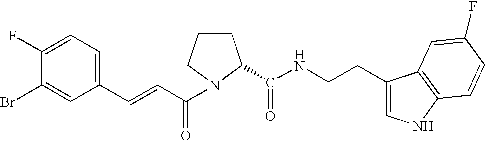 | 502.3 |
| 21 | (E)-1-acetyl-4-(3-(3-bromo-4-fluorophenyl)acrylamido)-N-(2-(5-fluoro-1H-indol-3-yl)ethyl)piperidine-4-carboxamide | 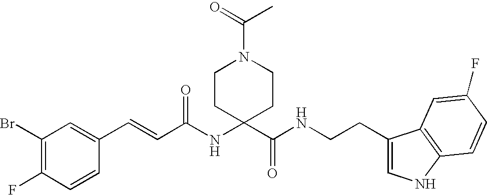 | 573.2 |
| 22 | (E)-4-(3-(3-bromo-4-fluorophenyl)acrylamido-N-(2-(5-fluoro-1H-indol-2-yl)ethyl)-1-methylpiperidine-4-carboxamide | 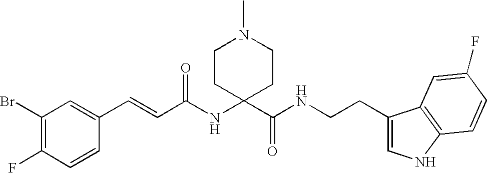 | 545.2 |
| 23 | (E)-methyl 4-(3-(3-bromo-4-fluorophenyl)acrylamido)-4-(2-(5-fluoro-1H-indol-3-yl)ethylcarbamoyl)piperidine-1-carboxylate | 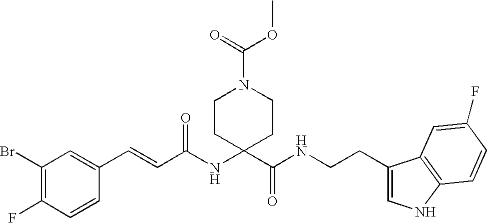 | 589.1 |
| 24 | (E)-1-(2-aminoacetyl)-4-(3-(3-bromo-4-fluorophenyl)acrylamido)-N-(2-(5-fluoro-1H-indol-3-yl)ethyl)piperidine-4-carboxamide | 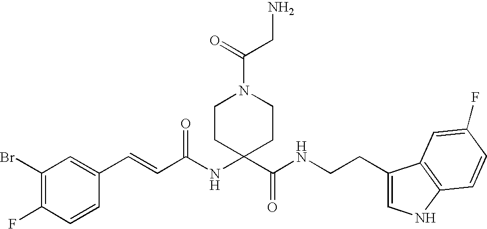 | 588.2 |
| 25 | (E)-4-(3-(3-bromo-4-fluorophenyl)acrylamido)-N-(2-(5-fluoro-1H-indol-3-yl)ethyl)-1-(3-methoxypropanoyl)piperidine-4-carboxamide | 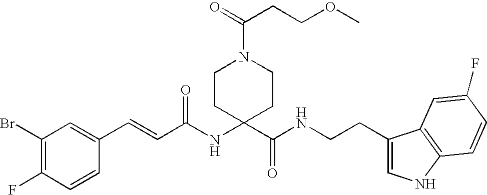 | 617.2 |
| 26 | (E)-4-(3-(3-bromo-4-fluorophenyl)acrylamido)-N1-ethyl-N4-(2-(5-fluoro-1H-indol-3-yl)ethyl)piperidine-1,4-dicarboxamide | 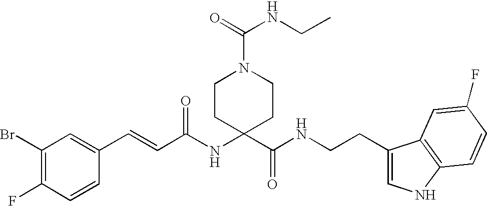 | 602.0 |
| 27 | (E)-4-(3-(3-bromo-4-fluorophenyl)acrylamido)-N-(2-(5-fluoro-1H-indol-3-yl)ethyl)-1-(2,2,2-trifluoroacetyl) piperidine-4-carboxamide | 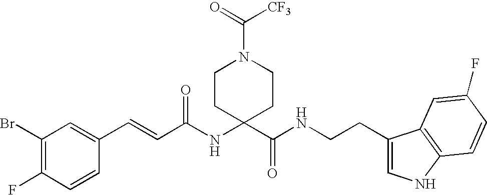 | 627.5 |
| 28 | (E)-1-(3-(3-bromo-4-fluorophenyl)acrylamido)-N1-(2-(5-fluoro-1H-indol-3-yl)ethyl)cyclohexane-1,4-dicarboxamide | 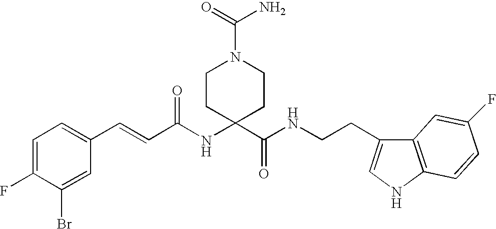 | 574.4 |
| 29 | (E)-(4-(3-(3-bromo-4-florophenyl)acrylamino)-1-carbamimidoyl-N-(2-(5-fluoro-1H-indol-3-yl)ethyl)piperidine-4-dicarboxamide | 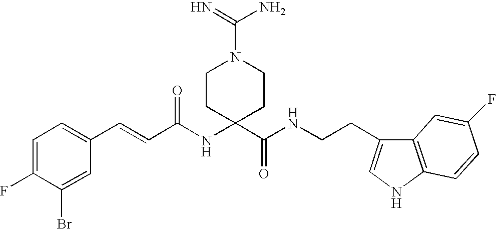 | 573.4 |
| 30 | (E)-4-(3-(3-bromo-4-fluorophenyl)-N-methyl acrylamido)-N-(2-(5-fluoro-1H-indol-3-yl) ethyl) piperidine-4-carboxamide | 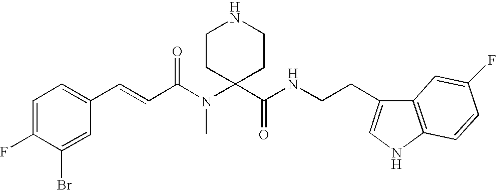 | 545.1 |
| 31 | (E)-3-(3,4-difluorophenyl)-N-(3-(dimethylamino)-1-(2-(5-fluoro-1H-indol-3-yl)ethylamino)-1-oxopropan-2-yl)acrylamide | 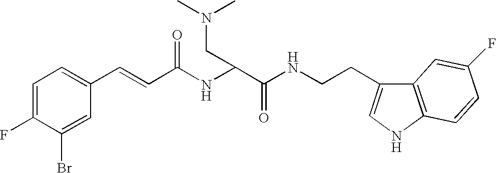 | 459.2 |
| 32 | (E)-N-(3,5-bis(trifluoromethyl)phenyl)-4-(3-(4-fluorophenyl)acrylamido)piperidine-4-carboxamide | 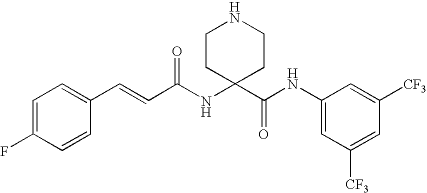 | 504.2 |
| 33 | (E)-4-(3-(4-fluorophenyl)acrylamido)-N-(3-(trifluoromethyl)phenyl)piperidine-4-carboxamide | 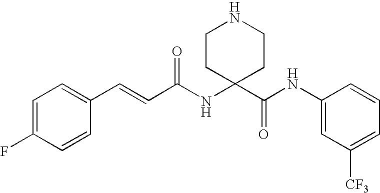 | 436.0 |
| 34 | (E)-4-(3-(3,4-difluorophenyl)acrylamido)-N-phenylpiperidine-4-carboxamide | 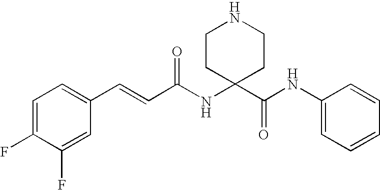 | 386.2 |
| 35 | (E)-4-(3-(3,4-difluorophenyl)acrylamido)-N-(4-phenoxyphenyl)piperidine-4-carboxamide | 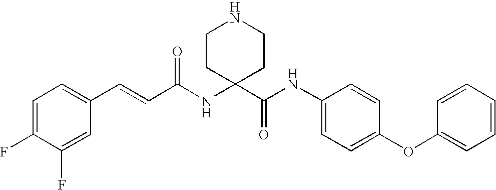 | 478.3 |
| 36 | (E)-N-(3,5-difluorophenyl)-4-(3-(4-fluorophenyl)acrylamido)piperidine-4-carboxamide | 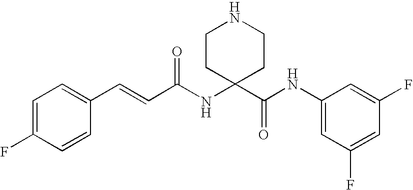 | 404.3 |
| 37 | (E)-N-(2-(1H-indol-3-yl)ethyl)-4-(3-(4-fluoro-3-(trifluoromethyl)phenyl)acrylamido)piperidine-4-carboxamide | 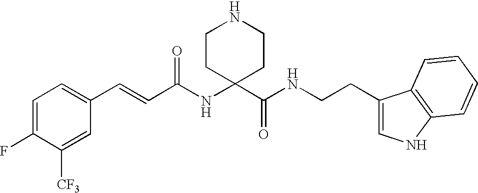 | 503.3 |
| 38 | (E)-N-(3,5-difluorophenyl)-4-(3-(3,4-difluorophenyl)acrylamido)piperidine-4-carboxamide | 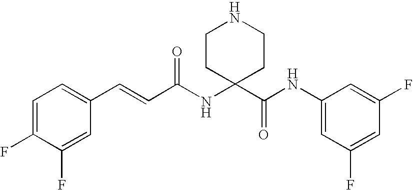 | 422.3 |
| 39 | (E)-4-(3-(4-fluorophenyl)acrylamido)-N-(4-(trifluoromethyl)phenyl)piperidine-4-carboxamide | 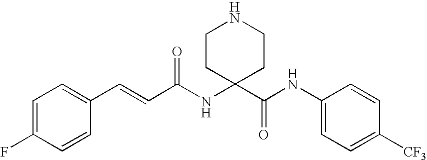 | 436.4 |
| 40 | (Z)-N-(3,5-bis(trifluoromethyl)phenyl)-4-(3-phenylacrylamido)piperidine-4-carboxamide | 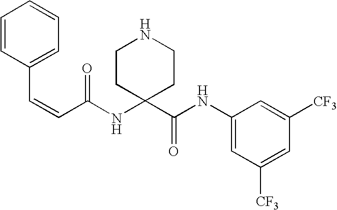 | 504.3 |
| 41 | N-(3,5-bis(trifluoromethyl)phenyl)-4-cinnamamidopiperidine-4-carboxamide | 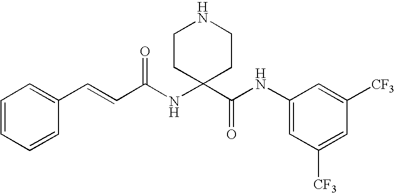 | 486.3 |
| 42 | (E)-N-(3,5-bis(trifluoromethyl)phenyl)-4-(3-(4-fluorophenyl)acrylamido)-1-methylpiperidine-4-carboxamide | 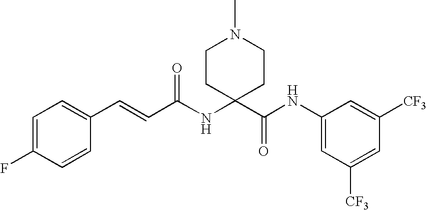 | 518.3 |
| 43 | (E)-N-(3,5-bis(trifluoromethyl)phenyl)-4-(3-(4-fluoro-3-methylphenyl)acrylamido)-1-methylpiperidine-4-carboxamide | 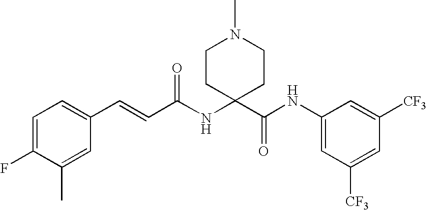 | 518.4 |
| 44 | (E)-N-(3,5-dichlorophenyl)-4-(3-(4-fluorophenyl)acrylamido)piperidine-4-carboxamide | 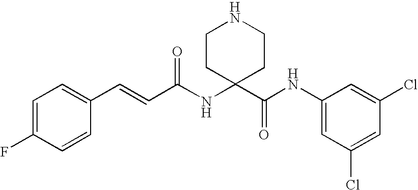 | 436.3 |
| 45 | (E)-N-(3,5-bis(trifluoromethyl)phenyl)-4-(3-(3,5-difluorophenyl)acrylamido)piperidine-4-carboxamide | 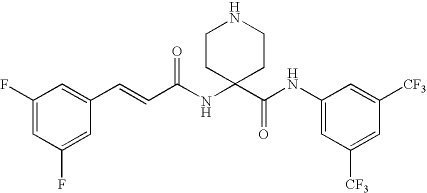 | 522.3 |
| 46 | (E)-4-(3-(4-fluorophenyl)acrylamido)-N-(2-(trifluoromethyl)phenyl)piperidine-4-carboxamide | 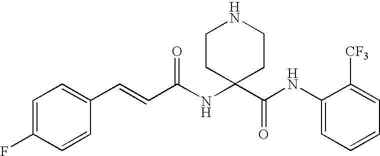 | 436.4 |
| 47 | (E)-4-(3-(4-fluoro-3-(trifluoromethyl)phenyl)acrylamido)-N-(3-(trifluoromethyl)phenyl)piperidine-4-carboxamide | 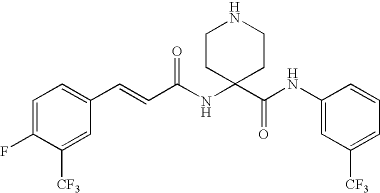 | 504.3 |
| 48 | (E)-N-(3,5-bis(trifluoromethyl)phenyl)-4-(3-(3-chloro-4-fluorophenyl)acrylamido)piperidine-4-carboxamide | 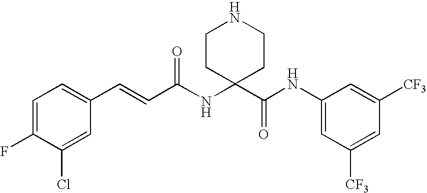 | 538.2 |
| 49 | (E)-N-(2-(5-fluoro-1H-indol-3-yl)ethyl)-4-(3-(4-fluorophenyl)acrylamido)-N-methylpiperidine-4-carboxamide | 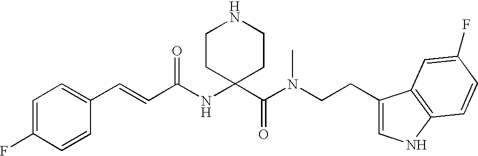 | 467.4 |
| 50 | (E)-4-(3-(3,4-difluorophenyl)acrylamido)-N-(2-phenoxyphenyl)piperidine-4-carboxamide | 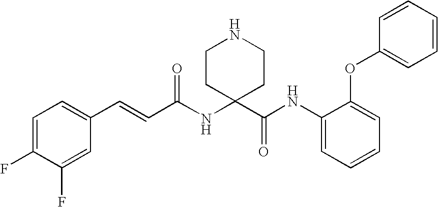 | 460.4 |
| 51 | (E)-4-(3-(3,4-difluorophenyl)acrylamido)-N-(3-phenoxyphenyl)piperidine-4-carboxamide | 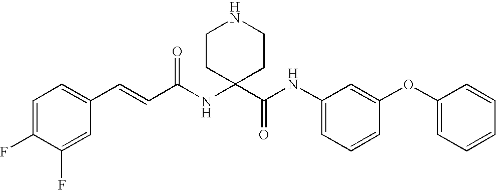 | 460.4 |
| 52 | (E)-N-(3,5-bis(trifluoromethyl)phenyl)-4-(3-(4-fluorophenyl)acrylamido)-1-(pyrimidin-2-yl)piperidine-4-carboxamide | 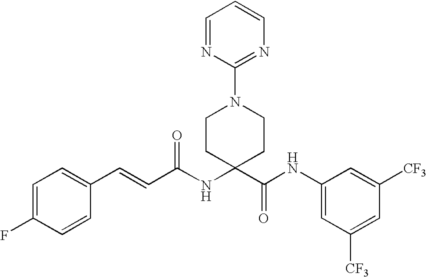 | 582.3 |
| 53 | (R,E)-N-(1-(3,5-bis(trifluoromethyl)phenyl)ethyl)-4-(3-(4-fluorophenyl)acrylamido)piperidine-4-carboxamide | 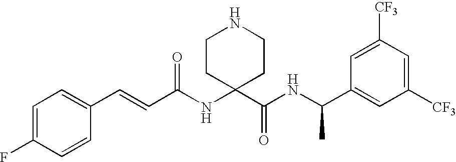 | 532.4 |
| 54 | (E)-N-(3,5-bis(trifluoromethyl)phenyl)-4-(3-(4-fluorophenyl)acrylamido)-1-(tetrahydro-2H-pyran-4-yl)piperidine-4-carboxamide | 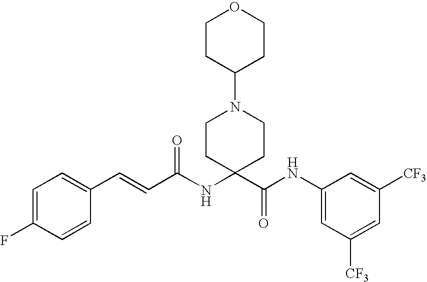 | 588.4 |
| 55 | (E)-N-(2-(3,5-dichlorophenylamino)-2-oxo-1-(pyridin-3-yl)ethyl)-3-(4-fluorophenyl)acrylamide | 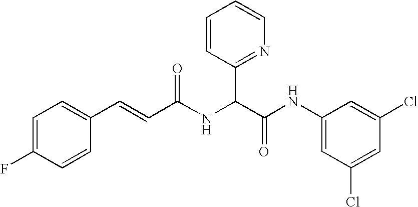 | 444.4 |
| 56 | (E)-N-(3,5-bis(trifluoromethyl)phenyl)-1-(3-(4-fluorophenyl)acrylamido)-4-hydroxycyclohexanecarboxamide | 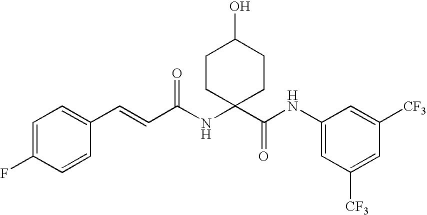 | 519.4 |
| 57 | (E)-N-(3,5-bis(trifluoromethyl)phenyl)-4-(3-(4-fluorophenyl)acrylamido)-1-(2-methoxyethyl)piperidine-4-carboxamide | 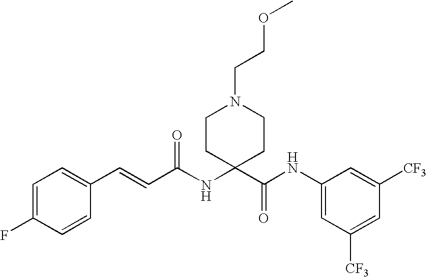 | 562.4 |
| 58 | (E)-N-(3,5-bis(trifluoromethyl)phenyl)-1-(3-(4-fluorophenyl)acrylamido)-4-morpholinocyclohexanecarboxamide | 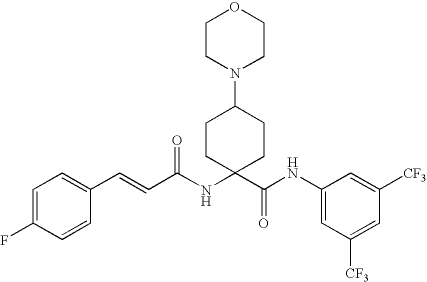 | 588.3 |
| 59 | (E)-N-(3,5-bis(trifluoromethyl)phenyl)-1-(3-(4-fluorophenyl)acrylamido)-4-(2-methoxyethylamino)cyclohexanecarboxamide | 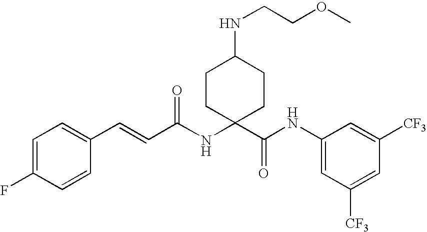 | 576.4 |
| 60 | (E)-N-(3,5-bis(trifluoromethyl)phenyl)-4-(3-(4-fluorophenyl)acrylamido)-1-(pyridin-2-yl)piperidine-4-carboxamide | 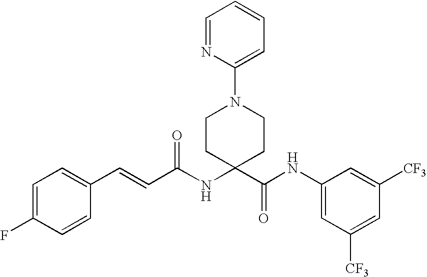 | 581.3 |
| 61 | (E)-N-(3,5-bis(trifluoromethyl)phenyl)-1-(3-(4-fluorophenyl)acrylamido)-4-(2-methoxyethoxy)cyclohexanecarboxamide | 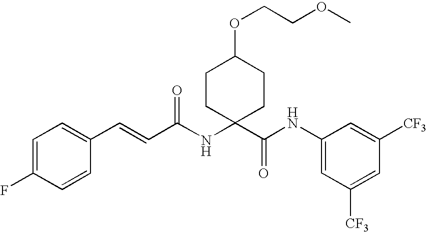 | 577.3 |
| 62 | (E)-1-(2-amino-2-oxoethyl)-N-(3,5-bis(trifluoromethyl)phenyl)-4-(3-(4-fluorophenyl)acrylamido)piperidine-4-carboxamide | 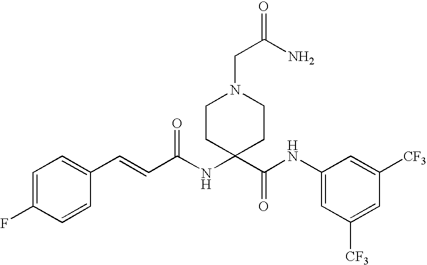 | 561.3 |
| 63 | (E)-1-(2-aminoacetyl)-N-(3,5-bis(trifluoromethyl)phenyl)-4-(3-(4-fluorophenyl)acrylamido)piperidine-4-carboxamide | 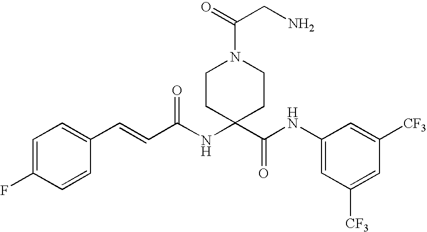 | 561.3 |
| 64 | (E)-N-(2-(5-fluoro-1H-indol-3-yl)ethyl)-4-(3-(4-fluorophenyl)acrylamido)-N-methyl-1-(tetrahydro-2H-pyran-4-yl)piperidine-4-carboxamide | 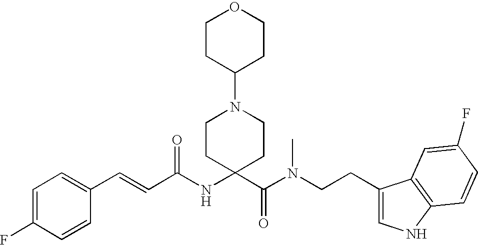 | 551.5 |
| 65 | (E)-1-(2-amino-2-oxoethyl)-N-(2-(5-fluoro-1H-indol-3-yl)ethyl)-4-(3-(4-fluorophenyl)acrylamido)-N-methylpiperidine-4-carboxamide | 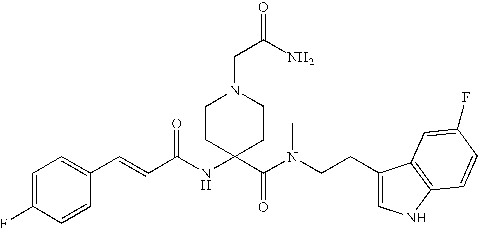 | 524.5 |
| 66 | (E)-N-(3,5-bis(trifluoromethyl)phenyl)-4-(3-(4-fluorophenyl)acrylamido)-1-(2-hydroxy-2-methylpropanoyl)piperidine-4-carboxamide | 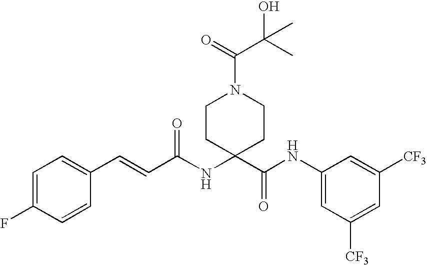 | 590.7 |
| 67 | (E)-1-(2-aminoacetyl)-N-(2-(5-fluoro-1H-indol-3-yl)ethyl)-4-(3-(4-fluorophenyl)acrylamido)-N-methylpiperidine-4-carboxamide | 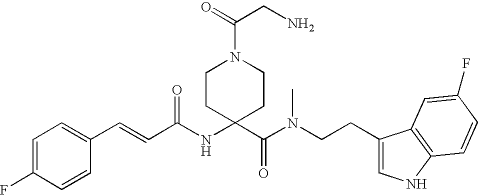 | 524.8 |
| 68 | (E)-4-(3-(3-bromo-4-fluorophenyl)acrylamido)-N-(2-(5-fluoro-1H-indol-3-yl)ethyl)-1-(2-hydroxy-2-methylpropyl)piperidine-4-carboxamide | 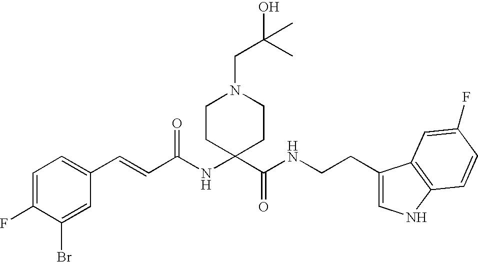 | 605.8 |
| 69 | (E)-N-(2-(5-fluoro-1H-indol-3-yl)ethyl)-4-(3-(4-fluorophenyl)acrylamido)-1-(2-hydroxy-2-methylpropyl)piperidine-4-carboxamide | 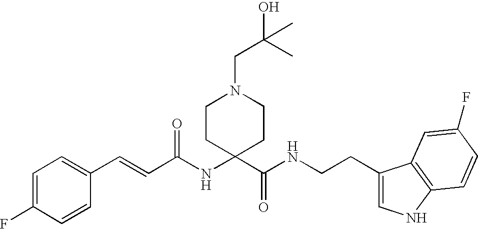 | 525.8 |
| 70 | (E)-N-(3,5-bis(trifluoromethyl)phenyl)-4-(3-(4-fluorophenyl)acrylamido)-1-(2-hydroxy-2-methylpropyl)piperidine-4-carboxamide | 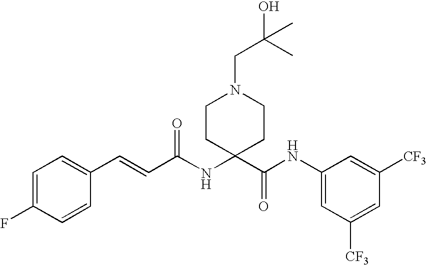 | 576.8 |
| 71 | (E)-N-(3,5-bis(trifluoromethyl)phenyl)-4-(3-(4-fluorophenyl)acrylamido)-1-(methylsulfonyl)piperidine-4-carboxamide | 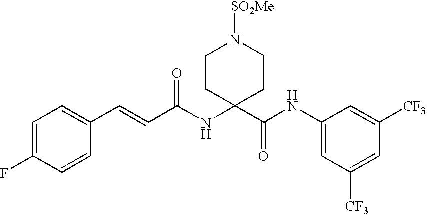 | 582.7 |
| 72 | (E)-N-(2-(5-fluoro-1H-indol-3-yl)ethyl)-4-(3-(4-fluorophenyl)acrylamido)-1-(2-hydroxy-2-methylpropanoyl)piperidine-4-carboxamide | 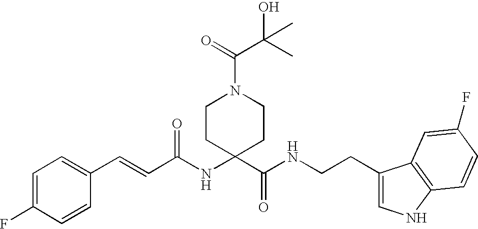 | 539.8 |
| 73 | (E)-1-(2-amino-2-oxoethyl)-N-(2-(5-fluoro-1H-indol-3-yl)ethyl)-4-(3-(4-fluorophenyl)acrylamido)piperidine-4-carboxamide | 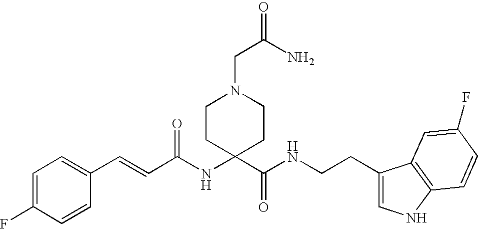 | 510.7 |
| 74 | (E)-4-(ethoxyimino)-N-(2-(5-fluoro-1H-indol-3-yl)ethyl)-1-(3-(4-fluorophenyl)acrylamido)-N-methylcyclohexanecarboxamide | 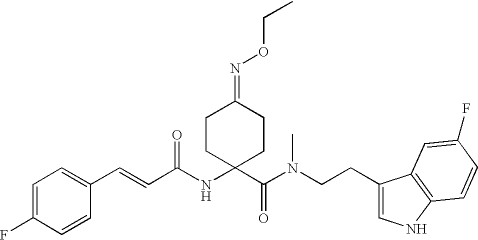 | 523.8 |

### EXAMPLE 17

### T-type Channel Blocking Activities of Various Invention Compounds

A. Transformation of HEK Cells:

T-type calcium channel blocking activity was assayed in human embryonic kidney cells, HEK 293, stably transfected with the T-type calcium channel subunits. Briefly, cells were cultured in Dulbecco's modified eagle medium (DMEM) supplemented with 10% fetal bovine serum, 200 U/ml penicillin and 0.2 mg/ml streptomycin at 37° C. with 5% CO₂. At 85% confluency cells were split with 0.25% trypsin/1 mM EDTA and plated at 10% confluency on glass coverslips. At 12 hours the medium was replaced and the cells stably transfected using a standard calcium phosphate protocol and the appropriate calcium channel cDNA's. Fresh DMEM was supplied and the cells transferred to 28° C./5% CO₂. Cells were incubated for 1 to 2 days prior to whole cell recording.

Standard patch-clamp techniques were employed to identify blockers of T-type currents. Briefly, previously described HEK cell lines stably expressing human α_{1G}, α_{1H} and α_{1I} T-type channels were used for all the recordings (passage #: 4-20, 37° C., 5% CO₂). Whole cell patch clamp experiments were performed using an Axopatch 200B amplifier (Axon Instruments, Burlingame, Calif.) linked to a personal computer equipped with pCLAMP software. Data were analyzed using Clampfit (Axon Instruments) and SigmaPlot 4.0 (Jandel Scientific). To obtain T-type currents, plastic dishes containing semi-confluent cells were positioned on the stage of a ZEISS AXIOVERT S100 microscope after replacing the culture medium with external solution (see below). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (see below for internal solution).

TABLE 2
External Solution 500 ml - pH 7.4, 265.5 mOsm

| Salt | Final mM | Stock M | Final ml |
|---|---|---|---|
| CsCl | 142 | 1 | 71 |
| CaCl₂ | 2 | 1 | 1 |
| MgCl₂ | 1 | 1 | 0.5 |
| HEPES | 10 | 0.5 | 10 |
| Glucose | 10 | — | 0.9 grams |

TABLE 3
Internal Solution 50 ml - pH 7.3 with CsOH, 270 mOsm

| Salt | Final mM | Stock M | Final ml |
|---|---|---|---|
| Cs-Methanesulfonate | 126.5 | — | 1.442 gr/50 ml |
| MgCl2 | 2 | 1 | 0.1 |
| HEPES | 10 | 0.5 | 1 |
| EGTA-Cs | 11 | 0.25 | 2.2 |
| ATP | 2 | 0.2 | 0.025 |
| | | | (1 aliquot/2.5 ml) |

T-type currents were reliably obtained by using two voltage protocols:
(1) “non-inactivating”, and
(2) “inactivation”

In the non-inactivating protocol, the holding potential is set at −110 mV and with a pre-pulse at −100 mV for 1 second prior to the test pulse at −40 mV for 50 ms. In the inactivation protocol, the pre-pulse is at approximately −85 mV for 1 second, which inactivates about 15% of the T-type channels.

Test compounds were dissolved in external solution, 0.1-0.01% DMSO. After 10 min rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. K_{d} values are shown for various compounds of the invention in Table 4 measured at 1 μM for the drug of interest except for compound 18 which was measured at 200 nM.

TABLE 4
T-type Calcium Channel Block

| Compound | α_{1G} (μM) | α_{1H} (μM) | α_{1I} (μM) |
|---|---|---|---|
| 1 | 0.22 | 0.47 | 0.18 |
| 2 | 0.03 | 0.41 | 0.03 |
| 7 | 0.13 | 137.00 | 0.11 |
| 12 | 0.12 | 1.80 | 0.28 |
| 13 | 0.14 | 1.79 | 0.39 |
| 14 | 0.29 | 12.00 | 0.46 |
| 15 | 0.36 | | 0.48 |
| 16 | 0.43 | | |
| 17 | 0.10 | 0.94 | 0.17 |
| 18 | 0.07 | 0.69 | 0.38 |
| 19 | 1.35 | | |
| 21 | 2.62 | | |
| 22 | 0.10 | 0.49 | 0.33 |
| 23 | 0.79 | | 0.87 |
| 24 | 0.18 | 1.45 | 0.44 |
| 25 | 5.37 | | |
| 26 | 0.55 | | |
| 27 | 0.15 | 0.84 | 0.21 |
| 32 | 0.05 | 0.42 | 0.05 |
| 33 | 2.45 | | |
| 34 | 3.29 | | |
| 40 | 0.13 | 2.42 | |
| 42 | 0.22 | 41.94 | |
| 43 | 0.08 | 1.12 | |
| 44 | 0.32 | 1.61 | |
| 46 | 14.61 | | |
| 49 | 0.58 | | |
| 50 | 0.08 | 2.97 | |
| 52 | 0.16 | | |
| 54 | 0.21 | 6.32 | |
| 55 | 0.21 | | |
| 56 | 1.06 | | |
| 57 | 0.13 | | |
| 61 | 0.99 | | |
| 62 | 0.28 | | |
| 63 | 0.09 | | |
| 65 | 2.57 | | |
| 66 | 1.63 | | |
| 67 | 1.02 | | |
| 69 | 3.63 | | |

1. A method to treat cardiovascular disease, epilepsy, diabetes, chronic or acute pain, sleep disorders, Parkinson's disease, psychosis, overactive bladder or male birth control, which method comprises administering to a subject in need of such treatment an amount of the compound of formula (1) effective to ameliorate said condition, wherein said compound is of the formula: 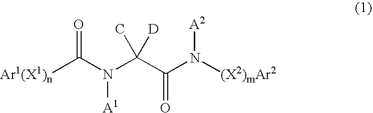
or a pharmaceutically acceptable salt or conjugate thereof, wherein
each X¹ and X² is independently an optionally substituted alkylene (1-3C), alkenylene (2-3C), alkynylene (2-3C), heteroalkylene (2-3C), heteroalkenylene (2-3C), or heteroalkynylene (2-3C);
Ar¹ is an optionally substituted phenyl ring;
Ar² is an optionally substituted aromatic (6-10 membered) or heteroaromatic (5-10 membered) ring;
each A¹ and A² are independently H or methyl;
C is an optionally substituted alkylene (1-3C), alkenylene (2-3C), alkynylene (2-3C), heteroalkylene (2-3C), heteroalkenylene (2-3C), heteroalkynylene (2-3C), aromatic (6-membered) or heteroaromatic (5-10 membered) ring;
D is H, or an optionally substituted alkylene (1-3C), alkenylene (2-3C), alkynylene (2-3C), heteroalkylene (2-3C), heteroalkenylene (2-3C), heteroalkynylene (2-3C),
wherein either C and A¹ or C and D may optionally together form an optionally substituted 3-6 membered cyclic or heterocyclic ring;
n and m are independently 0 or 1; and
wherein the optional substituents on each Ar¹, Ar², X¹, X², C and D are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-3C), alkenyl (2-3C), alkynyl (2-3C), heteroalkyl (2-3C) heteroalkenyl (2-3), and heteroalkynyl (2-3C); or the optional substituents may be one or more optionally substituted groups selected from alkyl (1-3C), alkenyl (2-3C), alkynyl (2-3C), heteroalkyl (2-3C), heteroalkenyl (2-3C), or heteroalkynyl (2-3C); and wherein the optional substituent on C and D may further be selected from ═O and ═NOR′;
and wherein optional substituents on a cyclic or heterocyclic ring formed with C and one of A¹ and D may independently be selected from ═O, ═NOR′, halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6) or heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), heteroalkynyl (2-6C), aromatic (6-10 membered) or heteroaromatic (6-10 membered) ring.

2. The method of claim 1 wherein said condition is cardiovascular disease, epilepsy, or chronic or acute pain.

3. The method of claim 1 wherein X¹ is an alkenylene (2-3C).

4. The method of claim 1 wherein X¹ is an alkenylene (2C).

5. The method of claim 1 wherein n is 1.

6. The method of claim 1 wherein optional substituents on Ar¹ are independently halo, methyl or CF₃.

7. The method of claim 1 wherein Ar² is an optionally substituted phenyl or indolyl ring.

8. The method of claim 7 wherein the optional substituents on Ar² are independently halo, methyl, CF₃, or phenoxy.

9. The method of claim 1 wherein C and D together form an optionally substituted 3-6 membered cyclic or heterocyclic ring.

10. The method of claim 9 wherein C and D together form an optionally substituted 6 membered cyclic or heterocyclic ring.

11. The method of claim 10 wherein C and D together form an optionally substituted piperidinyl ring.

12. The method of claim 9 wherein the optional substituents on a ring formed by C and D are independently COCH₃, OH, CH₂CH₂OH, CH₂OH, (CH₂)₂OCH₃, NH(CH₂)₂OCH₃, O(CH₂)₂OCH₃, CH₃, COOCH₃, COCH₂NH₂, CH₂CONH₂, CO(CH₂)₂OCH₃, CONHCH₂CH₃, COCF₃, CONH₂, C(NH)NH₂, CH₂CONH₂, COCH₂NH₂, CH₂CONH₂, COC(OH)(CH₃)₂, COCH₂NH₂, CH₂C(OH)(CH₃)₂, SO₂CH₃, ═NOCH₂CH₃, aromatic (6 membered) or heteroaromatic (5-6 membered) ring, or cyclic or heterocyclic (3-6 membered) ring.

13. The method of claim 1 wherein the compound is:
(E)-4-(3-(3-bromo-4-fluorophenyl)acrylamido)-N-(2-(5-fluoro-1H-indol-3-yl)ethyl)piperidine-4-carboxamide;
(E)-N-(2-(1H-indol-3-yl)ethyl)-4-(3-(3-bromo-4-fluorophenyl)acrylamido)piperidine-4-carboxamide;
(E)-4-(3-(3-bromophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-N-methylpiperidine-4-carboxamide;
(Z)-4-(3-(3-bromo-4-fluorophenyl)-3-fluoroacrylamido)-N-(2-(5-fluoro-1H-indol-3-yl)ethyl)piperidine-4-carboxamide;
(E)-4-(3-(3-bromo-4-fluorophenyl)acrylamido)-N-(2,4-difluorobenzyl)piperidine-4-carboxamide;
(E)-4-(3-(3,4-difluorophenyl)acrylamido)-N-(2-(5-fluoro-1H-indol-3-yl)ethyl)piperidine-4-carboxamide;
(E)-N-(2-(5-fluoro-1H-indol-3-yl)ethyl)-4-(3-(4-fluorophenyl)acrylamido)piperidine-4-carboxamide;
(E)-4-(3-(3-chloro-4-fluorophenyl)acrylamido)-N-(2-(5-fluoro-1H-indol-3-yl)ethyl)piperidine-4-carboxamide;
(E)-4-(3-(5-fluorophenyl)acrylamido)-N-(2-(5-fluoro-1H-indol-3-yl)ethyl)piperidine-4-carboxamide;
N-(2-(5-fluoro-1H-indol-3-yl)ethyl)-4-(3-(4-fluorophenyl)propanamido)piperidine-4-carboxamide;
(E)-1-(3-(3-bromo-4-fluorophenyl)acrylamido)-N-(2-(5-fluoro-1H-indol-3-yl)ethyl)cyclohexanecarboxamide;
(E)-1-(3-(3-bromo-4-fluorophenyl)acrylamido)-N-(2-(5-fluoro-1H-indol-3-yl)ethyl)cyclopentanecarboxamide;
(E)-1-(3-(3-bromo-4-fluorophenyl)acrylamido)-N-(2-(5-fluoro-1H-indol-3-yl)ethyl)cyclopropanecarboxamide;
(E)-3-(3-bromo-4-fluorophenyl)-N-(1-(2-(5-fluoro-1H-indol-3-yl)ethylamino)-2-methyl-1-oxopropan-2-yl)acrylamide;
(E)-4-(3-(3-bromo-4-fluorophenyl)acrylamido)-N-(2-(5-fluoro-1H-indol-3-yl)ethyl)tetrahydro-2H-pyran-4-carboxamide;
(S,E)-3-(3-bromo-4-fluorophenyl)-N-(2-(2-(5-fluoro-1H-indol-3-yl)ethylamino)-2-oxo-1-phenylethyl)acrylamide;
(R,E)-3-(3-bromo-4-fluorophenyl)-N-(2-(2-(5-fluoro-1H-indol-3-yl)ethylamino)-2-oxo-1-phenylethyl)acrylamide;
(R,E)-1-(3-(3-bromo-4-fluorophenyl)acryloyl)-N-(2-(5-fluoro-1H-indol-3-yl)ethyl)pyrrolidine-2-carboxamide;
(R,E)-1-(3-(3-bromo-4-fluorophenyl)acryloyl)-N-(2-(5-fluoro-1H-indol-3-yl)ethyl)pyrrolidine-2-carboxamide;
(E)-1-acetyl-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-methylpiperidine-4-carboxamide;
(E)-methyl 4-(3-(3-bromo-4-fluorophenyl)acrylamido)-4-(2-(5-fluoro-1H-indol-3-yl)ethylcarbamoyl)piperidine-1-carboxylate;
(E)-1-(2-aminoacetyl)-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-(3-methoxypropanol)piperidine-4-carboxamide;
(E)-4-(3-(3-bromo-4-fluorophenyl)acrylamido)-N1-ethyl-N4-(2-(5-fluoro-1H-indol-3-yl)ethyl)piperidine-1,4-dicarboxamide;
(E)-4-(3-(3-bromo-4-fluorophenyl)acrylamido)-N-(2-(5-fluoro-1H-indol-3-yl)ethyl)-1-(2,2,2-trifluoroacetyl)piperidine-4-carboxamide;
(E)-1-(3-(3-bromo-4-fluorophenyl)acrylamido)-N1-(2-(5-fluoro-1H-indol-3-yl)ethyl)cyclohexane-1,4-dicarboxamide;
(E)-(4-(3-(3-bromo-4-fluorophenyl)acrylamido)-1-carbamimidoyl-N-(2-(5-fluoro-1H-indol-3-yl)ethyl)piperidine-4-dicarboxamide;
(E)-4-(3-(3-bromo-4-fluorophenyl)-N-methyl acrylamido)-N-(2-(5-fluoro-1H-indol-3-yl)ethyl)piperidine-4-carboxamide;
(E)-3-(3,4-difluorophenyl)-N-(3-(dimethylamino)-1-(2-(5-fluoro-1H-indol-3-yl)ethylamino)-1-oxopropan-2-yl)acrylamide;
(E)-N-(3,5-bis(trifluoromethyl)phenyl)-4-(3-(4-fluorophenyl)acrylamido) piperidine-4-carboxamide;
(E)-4-(3-(4-fluorophenyl)acrylamido)-N-(3-(trifluoromethyl)phenyl)piperidine-4-carboxamide;
(E)-4-(3-(3,4-difluorophenyl)acrylamido)-N-phenylpiperidine-4-carboxamide;
(E)-4-(3-(3,4-difluorophenyl)acrylamido)-N-(4-phenoxyphenyl)piperidine-4-carboxamide;
(E)-N-(3,5-difluorophenyl)-4-(3-(4-fluorophenyl)acrylamido)piperidine-4-carboxamide;
(E)-N-(2-(1H-indol-3-yl)ethyl)-4-(3-(4-fluoro-3-(trifluoromethyl)phenyl)acrylamido)piperidine-4-carboxamide;
(E)-N-(3,5-difluorophenyl)-4-(3-(3,4-difluorophenyl)acrylamido)piperidine-4-carboxamide;
(E)-4-(3-(4-fluorophenyl)acrylamido)-N-(4-(trifluoromethyl)phenyl)piperidine-4-carboxamide;
(Z)-N-(3,5-bis(trifluoromethyl)phenyl)-4-(3-phenylacrylamido)piperidine-4-carboxamide;
N-(3,5-bis(trifluoromethyl)phenyl)-4-cinnamamidopiperidine-4-carboxamide;
(E)-N-(3,5-bis(trifluoromethyl)phenyl)-4-(3-(4-fluorophenyl)acrylamido)-1-methylpiperidine-4-carboxamide;
(E)-N-(3,5-bis(trifluoromethyl)phenyl)-4-(3-(4-fluoro-3-methylphenyl)acrylamido)-1-methylpiperidine-4-carboxamide;
(E)-N-(3,5-dichlorophenyl)-4-(3-(4-fluorophenyl)acrylamido)piperidine-4-carboxamide;
(E)-N-(3,5-bis(trifluoromethyl)phenyl)-4-(3-(3,5-difluorophenyl)acrylamido) piperidine-4-carboxamide;
(E)-4-(3-(4-fluorophenyl)acrylamido)-N-(2-(trifluoromethyl)phenyl)piperidine-4-carboxamide;
(E)-4-(3-(4-fluoro-3-(trifluoromethyl)phenyl)acrylamido)-N-(3-(trifluoromethyl)phenyl)piperidine-4-carboxamide;
(E)-N-(3,5-bis(trifluoromethyl)phenyl)-4-(3-(3-chloro-4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methylpiperidine-4-carboxamide;
(E)-4-(3-(3,4-difluorophenyl)acrylamido)-N-(2-phenoxyphenyl)piperidine-4-carboxamide;
(E)-4-(3-(3,4-difluorophenyl)acrylamido)-N-(3-phenoxyphenyl)piperidine-4-carboxamide;
(E)-N-(3,5-bis(trifluoromethyl)phenyl)-4-(3-(4-fluorophenyl)acrylamido)-1-(pyrimidin-2-yl)piperidine-4-carboxamide;
(R,E)-N-(1-(3,5-bis(trifluoromethyl)phenyl)ethyl)-4-(3-(4-fluorophenyl)acrylamido)piperidine-4-carboxamide;
(E)-N-(3,5-bis(trifluoromethyl)phenyl)-4-(3-(4-fluorophenyl)acrylamido)-1-(tetrahydro-2H-pyran-4-yl)piperidine-4-carboxamide;
(E)-N-(2-(3,5-dichlorophenylamino)-2-oxo-1-(pyridin-3-yl)ethyl)-3-(4-fluorophenyl)acrylamide;
(E)-N-(3,5-bis(trifluoromethyl)phenyl)-1-(3-(4-fluorophenyl)acrylamido)-4-hydroxycyclohexanecarboxamide;
(E)-N-(3,5-bis(trifluoromethyl)phenyl)-4-(3-(4-fluorophenyl)acrylamido)-1-(2-methoxyethyl)piperidine-4-carboxamide;
(E)-N-(3,5-bis(trifluoromethyl)phenyl)-1-(3-(4-fluorophenyl)acrylamido)-4-morpholinocyclohexanecarboxamide;
(E)-N-(3,5-bis(trifluoromethyl)phenyl)-1-(3-(4-fluorophenyl)acrylamido)-4-(2-methoxyethylamino)cyclohexanecarboxamide;
(E)-N-(3,5-bis(trifluoromethyl)phenyl)-4-(3-(4-fluorophenyl)acrylamido)-1-(pyridin-2-yl)piperidine-4-carboxamide;
(E)-N-(3,5-bis(trifluoromethyl)phenyl)-1-(3-(4-fluorophenyl)acrylamido)-4-(2-methoxyethoxy)cyclohexane carboxamide;
(E)-1-(2-amino-2-oxoethyl)-N-(3,5-bis(trifluoromethyl)phenyl)-4-(3-(4-fluorophenyl)acrylamido)piperidine-4-carboxamide;
(E)-1-(2-aminoacetyl)-N-(3,5-bis(trifluoromethyl)phenyl)-4-(3-(4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methyl-1-(tetrahydro-2H-pyran-4-yl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)-N-methylpiperidine-4-carboxamide;
(E)-N-(3,5-bis(trifluoromethyl)phenyl)-4-(3-(4-fluorophenyl)acrylamido)-1-(2-hydroxy-2-methylpropanoyl)piperidine-4-carboxamide;
(E)-1-(2-aminoacetyl)-N-(2-(5-fluoro-1H-indol-3-yl)ethyl)-4-(3-(4-fluorophenyl)acrylamido)-N-methylpiperidine-4-carboxamide;
(E)-4-(3-(3-bromo-4-fluorophenyl)acrylamido)-N-(2-(5-fluoro-1H-indol-3-yl)ethyl)-1-(2-hydroxy-2-methylpropyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(methylsulfonyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropanoyl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)piperidine-4-carboxamide;
(E)-4-(ethoxyimino)-N-(2-(5-fluoro-1H-indol-3-yl)ethyl)-1-(3-(4-fluorophenyl)acrylamido)-N-methylcyclohexanecarboxamide;
or a pharmaceutically acceptable salt of one of these.

14. A pharmaceutical composition comprising a compound of the formula: 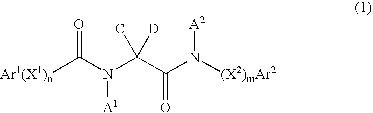
or a pharmaceutically acceptable salt or conjugate thereof in admixture with a pharmaceutically acceptable excipient, wherein
each X¹ and X² is independently an optionally substituted alkylene (1-3C), alkenylene (2-3C), alkynylene (2-3C), heteroalkylene (2-3C), heteroalkenylene (2-3C), or heteroalkynylene (2-3C);
Ar¹ is a phenyl ring optionally substituted with halo, methyl or CF₃;
Ar² is an optionally substituted aromatic (6-10 membered) or heteroaromatic (5-10 membered) ring;
each A¹ and A² are independently H or methyl;
C is an optionally substituted alkylene (1-3C), alkenylene (2-3C), alkynylene (2-3C), heteroalkylene (2-3C), heteroalkenylene (2-3C), heteroalkynylene (2-3C), aromatic (6-membered) or heteroaromatic (5-10 membered) ring;
D is H, or an optionally substituted alkylene (1-3C), alkenylene (2-3C), alkynylene (2-3C), heteroalkylene (2-3C), heteroalkenylene (2-3C), heteroalkynylene (2-3C),
wherein either C and A¹ or C and D may optionally together form an optionally substituted 3-6 membered cyclic or heterocyclic ring;
n and m are independently 0 or 1; and
wherein the optional substituents on each Ar², X¹, X², C and D are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-3C), alkenyl (2-3C), alkynyl (2-3C), heteroalkyl (2-3C) heteroalkenyl (2-3), and heteroalkynyl (2-3C); or the optional substituents may be one or more optionally substituted groups selected from alkyl (1-3C), alkenyl (2-3C), alkynyl (2-3C), heteroalkyl (2-3C), heteroalkenyl (2-3C), or heteroalkynyl (2-3C); and wherein the optional substituent on C and D may further be selected from ═O and ═NOR′;
and wherein optional substituents on a cyclic or heterocyclic ring formed with C and one of A¹ and D may independently be selected from ═O, ═NOR′, halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), and heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), heteroalkynyl (2-6C), aromatic (6-10 membered) or heteroaromatic (6-10 membered).

15. The pharmaceutical composition of claim 14 wherein X¹ is an alkenylene (2-3C).

16. The pharmaceutical composition of claim 14 wherein X¹ is an alkenylene (2C).

17. The pharmaceutical composition of claim 14 wherein n is 1.

18. The pharmaceutical composition of claim 14 wherein Ar₂ is an optionally substituted phenyl or indolyl ring.

19. The pharmaceutical composition of claim 18 wherein the optional substituents on Ar² are independently halo, methyl, CF₃, or phenoxy.

20. The pharmaceutical composition of claim 14 wherein C and D together form an optionally substituted 3-6 membered cyclic or heterocyclic ring.

21. The pharmaceutical composition of claim 20 wherein C and D together form an optionally substituted 6 membered cyclic or heterocyclic ring.

22. The pharmaceutical composition of claim 21 wherein C and D together form an optionally substituted piperidinyl ring.

23. The pharmaceutical composition of claim 20 wherein the optional substituents on a ring formed by C and D are independently COCH₃, OH, CH₂CH₂OH, CH₂OH, (CH₂)₂OCH₃, NH(CH₂)₂OCH₃, O(CH₂)₂OCH₃, CH₃, COOCH₃, COCH₂NH₂, CH₂CONH₂, CO(CH₂)₂OCH₃, CONHCH₂CH₃, COCF₃, CONH₂, C(NH)NH₂, CH₂CONH₂, COCH₂NH₂, CH₂CONH₂, COC(OH)(CH₃)₂, COCH₂NH₂, CH₂C(OH)(CH₃)₂, SO₂CH₃, ═NOCH₂CH₃, aromatic (6 membered) or heteroaromatic (5-6 membered) ring, or cyclic or heterocyclic (3-6 membered) ring.

24. The pharmaceutical composition of claim 14 wherein the compound is:
(E)-4-(3-(3-bromo-4-fluorophenyl)acrylamido)-N-(2-(5-fluoro-1H-indol-3-yl)ethyl)piperidine-4-carboxamide;
(E)-N-(2-(1H-indol-3-yl)ethyl)-4-(3-(3-bromo-4-fluorophenyl)acrylamido)piperidine-4-carboxamide;
(E)-4-(3-(3-bromophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-N-methylpiperidine-4-carboxamide;
(Z)-4-(3-(3-bromo-4-fluorophenyl)-3-fluoroacrylamido)-N-(2-(5-fluoro-1H-indol-3-yl)ethyl)piperidine-4-carboxamide;
(E)-4-(3-(3-bromo-4-fluorophenyl)acrylamido)-N-(2,4-difluorobenzyl)piperidine-4-carboxamide;
(E)-4-(3-(3,4-difluorophenyl)acrylamido)-N-(2-(5-fluoro-1H-indol-3-yl)ethyl)piperidine-4-carboxamide;
(E)-N-(2-(5-fluoro-1H-indol-3-yl)ethyl)-4-(3-(4-fluorophenyl)acrylamido)piperidine-4-carboxamide;
(E)-4-(3-(3-chloro-4-fluorophenyl)acrylamido)-N-(2-(5-fluoro-1H-indol-3-yl)ethyl)piperidine-4-carboxamide;
(E)-4-(3-(5-fluorophenyl)acrylamido)-N-(2-(5-fluoro-1H-indol-3-yl)ethyl)piperidine-4-carboxamide;
N-(2-(5-fluoro-1H-indol-3-yl)ethyl)-4-(3-(4-fluorophenyl)propanamido)piperidine-4-carboxamide;
(E)-1-(3-(3-bromo-4-fluorophenyl)acrylamido)-N-(2-(5-fluoro-1H-indol-3-yl)ethyl)cyclohexanecarboxamide;
(E)-1-(3-(3-bromo-4-fluorophenyl)acrylamido)-N-(2-(5-fluoro-1H-indol-yl)ethyl)cyclopentanecarboxamide;
(E)-1-(3-(3-bromo-4-fluorophenyl)acrylamido)-N-(2-(5-fluoro-1H-indol-yl)ethyl)cyclopropanecarboxamide;
(E)-3-(3-bromo-4-fluorophenyl)-N-(1-(2-(5-fluoro-1H-indol-3-yl)ethylamino)-2-methyl-1-oxopropan-2-yl)acrylamide;
(E)-4-(3-(3-bromo-4-fluorophenyl)acrylamido)-N-(2-(5-fluoro-1H-indol-3-yl)ethyl)tetrahydro-2H-pyran-4-carboxamide;
(S,E)-3-(3-bromo-4-fluorophenyl)-N-(2-(2-(5-fluoro-1H-indol-3-yl)ethylamino)-2-oxo-1-phenylethyl)acrylamide;
(R,E)-3-(3-bromo-4-fluorophenyl)-N-(2-(2-(5-fluoro-1H-indol-3-yl)ethylamino)-2-oxo-1-phenylethyl)acrylamide;
(R,E)-1-(3-(3-bromo-4-fluorophenyl)acryloyl)-N-(2-(5-fluoro-1H-indol-3-yl)ethyl)pyrrolidine-2-carboxamide;
(R,E)-1-(3-(3-bromo-4-fluorophenyl)acryloyl)-N-(2-(5-fluoro-1H-indol-3-yl)ethyl)pyrrolidine-2-carboxamide;
(E)-1-acetyl-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-methylpiperidine-4-carboxamide;
(E)-methyl 4-(3-(3-bromo-4-fluorophenyl)acrylamido)-4-(2-(5-fluoro-1H-indol-3-yl)ethylcarbamoyl)piperidine-1-carboxylate;
(E)-1-(2-aminoacetyl)-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-(3-methoxypropanol)piperidine-4-carboxamide;
(E)-4-(3-(3-bromo-4-fluorophenyl)acrylamido)-N1-ethyl-N4-(2-(5-fluoro-1H-indol-3-yl)ethyl)piperidine-1,4-dicarboxamide
(E)-4-(3-(3-bromo-4-fluorophenyl)acrylamido)-N-(2-(5-fluoro-1H-indol-3-yl)ethyl)-1-(2,2,2-trifluoroacetyl)piperidine-4-carboxamide;
(E)-1-(3-(3-bromo-4-fluorophenyl)acrylamido)-N1-(2-(5-fluoro-1H-indol-3-yl)ethyl)cyclohexane-1,4-dicarboxamide;
(E)-(4-(3-(3-bromo-4-fluorophenyl)acrylamido)-1-carbamimidoyl-N-(2-(5-fluoro-1H-indol-3-yl)ethyl)piperidine-4-dicarboxamide;
(E)-4-(3-(3-bromo-4-fluorophenyl)-N-methyl acrylamido)-N-(2-(5-fluoro-1H-indol-3-yl)ethyl)piperidine-4-carboxamide;
(E)-3-(3,4-difluorophenyl)-N-(3-(dimethylamino)-1-(2-(5-fluoro-1H-indol-3-yl)ethylamino)-1-oxopropan-2-yl)acrylamide;
(E)-N-(3,5-bis(trifluoromethyl)phenyl)-4-(3-(4-fluorophenyl)acrylamido) piperidine-4-carboxamide;
(E)-4-(3-(4-fluorophenyl)acrylamido)-N-(3-(trifluoromethyl)phenyl)piperidine-4-carboxamide;
(E)-4-(3-(3,4-difluorophenyl)acrylamido)-N-phenylpiperidine-4-carboxamide;
(E)-4-(3-(3,4-difluorophenyl)acrylamido)-N-(4-phenoxyphenyl)piperidine-4-carboxamide;
(E)-N-(3,5-difluorophenyl)-4-(3-(4-fluorophenyl)acrylamido)piperidine-4-carboxamide;
(E)-N-(2-(1H-indol-3-yl)ethyl)-4-(3-(4-fluoro-3-(trifluoromethyl)phenyl)acrylamido)piperidine-4-carboxamide;
(E)-N-(3,5-difluorophenyl)-4-(3-(3,4-difluorophenyl)acrylamido)piperidine-4-carboxamide;
(E)-4-(3-(4-fluorophenyl)acrylamido)-N-(4-(trifluoromethyl)phenyl)piperidine-4-carboxamide;
(Z)-N-(3,5-bis(trifluoromethyl)phenyl)-4-(3-phenylacrylamido)piperidine-4-carboxamide;
N-(3,5-bis(trifluoromethyl)phenyl)-4-cinnamamidopiperidine-4-carboxamide;
(E)-N-(3,5-bis(trifluoromethyl)phenyl)-4-(3-(4-fluorophenyl)acrylamido)-1-methylpiperidine-4-carboxamide;
(E)-N-(3,5-bis(trifluoromethyl)phenyl)-4-(3-(4-fluoro-3-methylphenyl)acrylamido)-1-methylpiperidine-4-carboxamide;
(E)-N-(3,5-dichlorophenyl)-4-(3-(4-fluorophenyl)acrylamido)piperidine-4-carboxamide;
(E)-N-(3,5-bis(trifluoromethyl)phenyl)-4-(3-(3,5-difluorophenyl)acrylamido)piperidine-4-carboxamide;
(E)-4-(3-(4-fluorophenyl)acrylamido)-N-(2-(trifluoromethyl)phenyl)piperidine-4-carboxamide;
(E)-4-(3-(4-fluoro-3-(trifluoromethyl)phenyl)acrylamido)-N-(3-(trifluoromethyl)phenyl)piperidine-4-carboxamide;
(E)-N-(3,5-bis(trifluoromethyl)phenyl)-4-(3-(3-chloro-4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methylpiperidine-4-carboxamide;
(E)-4-(3-(3,4-difluorophenyl)acrylamido)-N-(2-phenoxyphenyl)piperidine-4-carboxamide;
(E)-4-(3-(3,4-difluorophenyl)acrylamido)-N-(3-phenoxyphenyl)piperidine-4-carboxamide;
(E)-N-(3,5-bis(trifluoromethyl)phenyl)-4-(3-(4-fluorophenyl)acrylamido)-1-(pyrimidin-2-yl)piperidine-4-carboxamide;
(R,E)-N-(1-(3,5-bis(trifluoromethyl)phenyl)ethyl)-4-(3-(4-fluorophenyl)acrylamido)piperidine-4-carboxamide;
(E)-N-(3,5-bis(trifluoromethyl)phenyl)-4-(3-(4-fluorophenyl)acrylamido)-1-(tetrahydro-2H-pyran-4-yl)piperidine-4-carboxamide;
(E)-N-(2-(3,5-dichlorophenylamino)-2-oxo-1-(pyridin-3-yl)ethyl)-3-(4-fluorophenyl)acrylamide;
(E)-N-(3,5-bis(trifluoromethyl)phenyl)-1-(3-(4-fluorophenyl)acrylamido)-4-hydroxycyclohexanecarboxamide;
(E)-N-(3,5-bis(trifluoromethyl)phenyl)-4-(3-(4-fluorophenyl)acrylamido)-1-(2-methoxyethyl)piperidine-4-carboxamide;
(E)-N-(3,5-bis(trifluoromethyl)phenyl)-1-(3-(4-fluorophenyl)acrylamido)-4-morpholinocyclohexanecarboxamide;
(E)-N-(3,5-bis(trifluoromethyl)phenyl)-1-(3-(4-fluorophenyl)acrylamido)-4-(2-methoxyethylamino)cyclohexanecarboxamide;
(E)-N-(3,5-bis(trifluoromethyl)phenyl)-4-(3-(4-fluorophenyl)acrylamido)-1-(pyridin-2-yl)piperidine-4-carboxamide;
(E)-N-(3,5-bis(trifluoromethyl)phenyl)-1-(3-(4-fluorophenyl)acrylamido)-4-(2-methoxyethoxy)cyclohexane carboxamide;
(E)-1-(2-amino-2-oxoethyl)-N-(3,5-bis(trifluoromethyl)phenyl)-4-(3-(4-fluorophenyl)acrylamido)piperidine-4-carboxamide;
(E)-1-(2-aminoacetyl)-N-(3,5-bis(trifluoromethyl)phenyl)-4-(3-(4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methyl-1-(tetrahydro-2H-pyran-4-yl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)-N-methylpiperidine-4-carboxamide;
(E)-N-(3,5-bis(trifluoromethyl)phenyl)-4-(3-(4-fluorophenyl)acrylamido)-1-(2-hydroxy-2-methylpropanoyl)piperidine-4-carboxamide;
(E)-1-(2-aminoacetyl)-N-(2-(5-fluoro-1H-indol-3-yl)ethyl)-4-(3-(4-fluorophenyl)acrylamido)-N-methylpiperidine-4-carboxamide;
(E)-4-(3-(3-bromo-4-fluorophenyl)acrylamido)-N-(2-(5-fluoro-1H-indol-3-yl)ethyl)-1-(2-hydroxy-2-methylpropyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(methylsulfonyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropanoyl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)piperidine-4-carboxamide;
(E)-4-(ethoxyimino)-N-(2-(5-fluoro-1H-indol-3-yl)ethyl)-1-(3-(4-fluorophenyl)acrylamido)-N-methylcyclohexanecarboxamide;
or a pharmaceutically acceptable salt of one of these.

25. A compound of the formula: 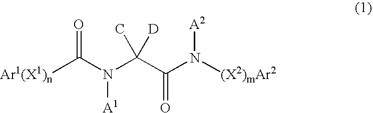
or a pharmaceutically acceptable salt or conjugate thereof, wherein
each X¹ and X² is independently an optionally substituted alkylene (1-3C), alkenylene (2-3C), alkynylene (2-3C), heteroalkylene (2-3C), heteroalkenylene (2-3C), or heteroalkynylene (2-3C);
Ar¹ is a phenyl ring optionally substituted with halo, methyl or CF₃;
Ar² is an optionally substituted aromatic (6-10 membered) or heteroaromatic (5-10 membered) ring;
each A¹ and A² are independently H or methyl;
C is an optionally substituted alkylene (1-3C), alkenylene (2-3C), alkynylene (2-3C), heteroalkylene (2-3C), heteroalkenylene (2-3C), heteroalkynylene (2-3C), aromatic (6-membered) or heteroaromatic (5-10 membered) ring;
D is H, or an optionally substituted alkylene (1-3C), alkenylene (2-3C), alkynylene (2-3C), heteroalkylene (2-3C), heteroalkenylene (2-3C), heteroalkynylene (2-3C),
wherein either C and A¹ or C and D may optionally together form an optionally substituted 3-6 membered cyclic or heterocyclic ring;
n and m are independently 0 or 1; and
wherein the optional substituents on each Ar¹, Ar², X¹, X², C and D are independently selected from halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-3C), alkenyl (2-3C), alkynyl (2-3C), heteroalkyl (2-3C) heteroalkenyl (2-3), and heteroalkynyl (2-3C); or the optional substituents may be one or more optionally substituted groups selected from alkyl (1-3C), alkenyl (2-3C), alkynyl (2-3C), heteroalkyl (2-3C), heteroalkenyl (2-3C), or heteroalkynyl (2-3C); and wherein the optional substituent on C and D may further be selected from ═O and ═NOR′;
and wherein optional substituents on a cyclic or heterocyclic ring formed with C and one of A¹ and D may independently be selected from ═O, ═NOR′, halo, CN, NO₂, CF₃, OCF₃, COOR′, CONR′₂, OR′, SR′, SOR′, SO₂R′, NR′₂, NR′(CO)R′, and NR′SO₂R′, wherein each R′ is independently H or an optionally substituted group selected from alkyl (1-6C), alkenyl (2-6C), alkynyl (2-6C), heteroalkyl (2-6C) heteroalkenyl (2-6), and heteroalkynyl (2-6C); or the optional substituents may be one or more optionally substituted groups selected from alkyl (1-6C), alkenyl (2-6C), alkynyl (2-6C), heteroalkyl (2-6C), heteroalkenyl (2-6C), or heteroalkynyl (2-6C), aromatic (6-10 membered) or heteroaromatic (6-10 membered);
with the proviso that the compound is not (E)-4-(3-(3-bromo-4-fluorophenyl)acrylamido)-N-(2-(5-fluoro-1H-indol-3-yl)ethyl)piperidine-4-carboxamide.

26. The compound of claim 25 wherein X¹ is an alkenylene (2-3C).

27. The compound of claim 25 wherein X¹ is an alkenylene (2C).

28. The compound of claim 25 wherein n is 1.

29. The compound of claim 25 wherein Ar² is an optionally substituted phenyl or indolyl ring.

30. The compound of claim 29 wherein the optional substituents on Ar² are independently halo, methyl, CF₃, or phenoxy.

31. The compound of claim 25 wherein C and D together form an optionally substituted 3-6 membered cyclic or heterocyclic ring.

32. The compound of claim 31 wherein C and D together form an optionally substituted 6 membered cyclic or heterocyclic ring.

33. The compound of claim 32 wherein C and D together form an optionally substituted piperidinyl ring.

34. The compound of claim 31 wherein the optional substituents on a ring formed by C and D are independently COCH₃, OH, CH₂CH₂OH, CH₂OH, (CH₂)₂OCH₃, NH(CH₂)₂OCH₃, O(CH₂)₂OCH₃, CH₃, COOCH₃, COCH₂NH₂, CH₂CONH₂, CO(CH₂)₂OCH₃, CONHCH₂CH₃, COCF₃, CONH₂, C(NH)NH₂, CH₂CONH₂, COCH₂NH₂, CH₂CONH₂, COC(OH)(CH₃)₂, COCH₂NH₂, CH₂C(OH)(CH₃)₂, SO₂CH₃, ═NOCH₂CH₃, aromatic (6 membered) or heteroaromatic (5-6 membered) ring, or cyclic or heterocyclic (3-6 membered) ring.

35. The compound of claim 25 wherein the compound is:
(E)-N-(2-(1H-indol-3-yl)ethyl)-4-(3-(3-bromo-4-fluorophenyl)acrylamido)piperidine-4-carboxamide;
(E)-4-(3-(3-bromophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-N-methylpiperidine-4-carboxamide;
(Z)-4-(3-(3-bromo-4-fluorophenyl)-3-fluoroacrylamido)-N-(2-(5-fluoro-1H-indol-3-yl)ethyl)piperidine-4-carboxamide;
(E)-4-(3-(3-bromo-4-fluorophenyl)acrylamido)-N-(2,4-difluorobenzyl)piperidine-4-carboxamide;
(E)-4-(3-(3,4-difluorophenyl)acrylamido)-N-(2-(5-fluoro-1H-indol-3-yl)ethyl)piperidine-4-carboxamide;
(E)-N-(2-(5-fluoro-1H-indol-3-yl)ethyl)-4-(3-(4-fluorophenyl)acrylamido)piperidine-4-carboxamide;
(E)-4-(3-(3-chloro-4-fluorophenyl)acrylamido)-N-(2-(5-fluoro-1H-indol-3-yl)ethyl)piperidine-4-carboxamide;
(E)-4-(3-(5-fluorophenyl)acrylamido)-N-(2-(5-fluoro-1H-indol-3-yl)ethyl)piperidine-4-carboxamide;
N-(2-(5-fluoro-1H-indol-3-yl)ethyl)-4-(3-(4-fluorophenyl)propanamido)piperidine-4-carboxamide;
(E)-1-(3-(3-bromo-4-fluorophenyl)acrylamido)-N-(2-(5-fluoro-1H-indol-3-yl)ethyl)cyclohexanecarboxamide;
(E)-1-(3-(3-bromo-4-fluorophenyl)acrylamido)-N-(2-(5-fluoro-1H-indol-3-yl)ethyl)cyclopentanecarboxamide;
(E)-1-(3-(3-bromo-4-fluorophenyl)acrylamido)-N-(2-(5-fluoro-1H-indol-3-yl)ethyl)cyclopropanecarboxamide;
(E)-3-(3-bromo-4-fluorophenyl)-N-(1-(2-(5-fluoro-1H-indol-3-yl)ethylamino)-2-methyl-1-oxopropan-2-yl)acrylamide;
(E)-4-(3-(3-bromo-4-fluorophenyl)acrylamido)-N-(2-(5-fluoro-1H-indol-3-yl)ethyl)tetrahydro-2H-pyran-4-carboxamide;
(S,E)-3-(3-bromo-4-fluorophenyl)-N-(2-(2-(5-fluoro-1H-indol-3-yl)ethylamino)-2-oxo-1-phenylethyl)acrylamide;
(R,E)-3-(3-bromo-4-fluorophenyl)-N-(2-(2-(5-fluoro-1H-indol-3-yl)ethylamino)-2-oxo-1-phenylethyl)acrylamide;
(R,E)-1-(3-(3-bromo-4-fluorophenyl)acryloyl)-N-(2-(5-fluoro-1H-indol-3-yl)ethyl)pyrrolidine-2-carboxamide;
(R,E)-1-(3-(3-bromo-4-fluorophenyl)acryloyl)-N-(2-(5-fluoro-1H-indol-3-yl)ethyl)pyrrolidine-2-carboxamide;
(E)-1-acetyl-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-methylpiperidine-4-carboxamide;
(E)-methyl 4-(3-(3-bromo-4-fluorophenyl)acrylamido)-4-(2-(5-fluoro-1H-indol-3-yl)ethylcarbamoyl)piperidine-1-carboxylate;
(E)-1-(2-aminoacetyl)-4-(3-(3-bromo-4-fluorophenyl)acrylamido)-N-(2-(5-fluoro-1H-indol-3-yl)ethyl)piperidine-4-carboxamide;
(E)-4-(3-(3-bromo-4-fluorophenyl)acrylamido)-N-(2-(5-fluoro-1H-indol-3-yl)ethyl)-1-(3-methoxypropanol)piperidine-4-carboxamide;
(E)-4-(3-(3-bromo-4-fluorophenyl)acrylamido)-N1-ethyl-N4-(2-(5-fluoro-1H-indol-3-yl)ethyl)piperidine-1,4-dicarboxamide;
(E)-4-(3-(3-bromo-4-fluorophenyl)acrylamido)-N-(2-(5-fluoro-1H-indol-3-yl)ethyl)-1-(2,2,2-trifluoroacetyl)piperidine-4-carboxamide;
(E)-1-(3-(3-bromo-4-fluorophenyl)acrylamido)-N1-(2-(5-fluoro-1H-indol-3-yl)ethyl)cyclohexane-1,4-dicarboxamide;
(E)-(4-(3-(3-bromo-4-fluorophenyl)acrylamido)-1-carbamimidoyl-N-(2-(5-fluoro-1H-indol-3-yl)ethyl)piperidine-4-dicarboxamide;
(E)-4-(3-(3-bromo-4-fluorophenyl)-N-methyl acrylamido)-N-(2-(5-fluoro-1H-indol-3-yl)ethyl)piperidine-4-carboxamide;
(E)-3-(3,4-difluorophenyl)-N-(3-(dimethylamino)-1-(2-(5-fluoro-1H-indol-3-yl)ethylamino)-1-oxopropan-2-yl)acrylamide;
(E)-N-(3,5-bis(trifluoromethyl)phenyl)-4-(3-(4-fluorophenyl)acrylamido) piperidine-4-carboxamide;
(E)-4-(3-(4-fluorophenyl)acrylamido)-N-(3-(trifluoromethyl)phenyl)piperidine-4-carboxamide;
(E)-4-(3-(3,4-difluorophenyl)acrylamido)-N-phenylpiperidine-4-carboxamide;
(E)-4-(3-(3,4-difluorophenyl)acrylamido)-N-(4-phenoxyphenyl)piperidine-4-carboxamide;
(E)-N-(3,5-difluorophenyl)-4-(3-(4-fluorophenyl)acrylamido)piperidine-4-carboxamide;
(E)-N-(2-(1H-indol-3-yl)ethyl)-4-(3-(4-fluoro-3-(trifluoromethyl)phenyl)acrylamido)piperidine-4-carboxamide;
(E)-N-(3,5-difluorophenyl)-4-(3-(3,4-difluorophenyl)acrylamido)piperidine-4-carboxamide;
(E)-4-(3-(4-fluorophenyl)acrylamido)-N-(4-(trifluoromethyl)phenyl)piperidine-4-carboxamide;
(Z)-N-(3,5-bis(trifluoromethyl)phenyl)-4-(3-phenylacrylamido)piperidine-4-carboxamide;
N-(3,5-bis(trifluoromethyl)phenyl)-4-cinnamamidopiperidine-4-carboxamide;
(E)-N-(3,5-bis(trifluoromethyl)phenyl)-4-(3-(4-fluorophenyl)acrylamido)-1-methylpiperidine-4-carboxamide;
(E)-N-(3,5-bis(trifluoromethyl)phenyl)-4-(3-(4-fluoro-3-methylphenyl)acrylamido)-1-methylpiperidine-4-carboxamide;
(E)-N-(3,5-dichlorophenyl)-4-(3-(4-fluorophenyl)acrylamido)piperidine-4-carboxamide;
(E)-N-(3,5-bis(trifluoromethyl)phenyl)-4-(3-(3,5-difluorophenyl)acrylamido) piperidine-4-carboxamide;
(E)-4-(3-(4-fluorophenyl)acrylamido)-N-(2-(trifluoromethyl)phenyl)piperidine-4-carboxamide;
(E)-4-(3-(4-fluoro-3-(trifluoromethyl)phenyl)acrylamido)-N-(3-(trifluoromethyl)phenyl)piperidine-4-carboxamide;
(E)-N-(3,5-bis(trifluoromethyl)phenyl)-4-(3-(3-chloro-4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methylpiperidine-4-carboxamide;
(E)-4-(3-(3,4-difluorophenyl)acrylamido)-N-(2-phenoxyphenyl)piperidine-4-carboxamide;
(E)-4-(3-(3,4-difluorophenyl)acrylamido)-N-(3-phenoxyphenyl)piperidine-4-carboxamide;
(E)-N-(3,5-bis(trifluoromethyl)phenyl)-4-(3-(4-fluorophenyl)acrylamido)-1-(pyrimidin-2-yl)piperidine-4-carboxamide;
(R,E)-N-(1-(3,5-bis(trifluoromethyl)phenyl)ethyl)-4-(3-(4-fluorophenyl)acrylamido)piperidine-4-carboxamide;
(E)-N-(3,5-bis(trifluoromethyl)phenyl)-4-(3-(4-fluorophenyl)acrylamido)-1-(tetrahydro-2H-pyran-4-yl)piperidine-4-carboxamide;
(E)-N-(2-(3,5-dichlorophenylamino)-2-oxo-1-(pyridin-3-yl)ethyl)-3-(4-fluorophenyl)acrylamide;
(E)-N-(3,5-bis(trifluoromethyl)phenyl)-1-(3-(4-fluorophenyl)acrylamido)-4-hydroxycyclohexanecarboxamide;
(E)-N-(3,5-bis(trifluoromethyl)phenyl)-4-(3-(4-fluorophenyl)acrylamido)-1-(2-methoxyethyl)piperidine-4-carboxamide;
(E)-N-(3,5-bis(trifluoromethyl)phenyl)-1-(3-(4-fluorophenyl)acrylamido)-4-morpholinocyclohexanecarboxamide;
(E)-N-(3,5-bis(trifluoromethyl)phenyl)-1-(3-(4-fluorophenyl)acrylamido)-4-(2-methoxyethylamino)cyclohexanecarboxamide;
(E)-N-(3,5-bis(trifluoromethyl)phenyl)-4-(3-(4-fluorophenyl)acrylamido)-1-(pyridin-2-yl)piperidine-4-carboxamide;
(E)-N-(3,5-bis(trifluoromethyl)phenyl)-1-(3-(4-fluorophenyl)acrylamido)-4-(2-methoxyethoxy)cyclohexane carboxamide;
(E)-1-(2-amino-2-oxoethyl)-N-(3,5-bis(trifluoromethyl)phenyl)-4-(3-(4-fluorophenyl)acrylamido)piperidine-4-carboxamide;
(E)-1-(2-aminoacetyl)-N-(3,5-bis(trifluoromethyl)phenyl)-4-(3-(4-fluorophenyl)acrylamido)piperidine-4-carboxamide;
(E)-N-(2-(5-fluoro-1H-indol-3-yl)ethyl)-4-(3-(4-fluorophenyl)acrylamido)-N-methyl-1-(tetrahydro-2H-pyran-4-yl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)-N-methylpiperidine-4-carboxamide;
(E)-N-(3,5-bis(trifluoromethyl)phenyl)-4-(3-(4-fluorophenyl)acrylamido)-1-(2-hydroxy-2-methylpropanoyl)piperidine-4-carboxamide;
(E)-1-(2-aminoacetyl)-N-(2-(5-fluoro-1H-indol-3-yl)ethyl)-4-(3-(4-fluorophenyl)acrylamido)-N-methylpiperidine-4-carboxamide;
(E)-4-(3-(3-bromo-4-fluorophenyl)acrylamido)-N-(2-(5-fluoro-1H-indol-3-yl)ethyl)-1-(2-hydroxy-2-methylpropyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(2-hydroxy-2-methylpropyl)piperidine-4-carboxamide;
(E)-N-(3,5-bis(trifluoromethyl)phenyl)-4-(3-(4-fluorophenyl)acrylamido)-1-(methylsulfonyl)piperidine-4-carboxamide;
(E)-N-(2-(5-fluoro-1H-indol-3-yl)ethyl)-4-(3-(4-fluorophenyl)acrylamido)-1-(2-hydroxy-2-methylpropanoyl)piperidine-4-carboxamide;
(E)-1-(2-amino-2-oxoethyl)-N-(2-(5-fluoro-1H-indol-3-yl)ethyl)-4-(3-(4-fluorophenyl)acrylamido)piperidine-4-carboxamide;
(E)-4-(ethoxyimino)-N-(2-(5-fluoro-1H-indol-3-yl)ethyl)-1-(3-(4-fluorophenyl)acrylamido)-N-methylcyclohexanecarboxamide;
or a pharmaceutically acceptable salt of one of these.
